(19) United States
(12) Patent Application Publication
(10) Pub. No.: US 2026/0294791 A1
(43) Pub. Date: Oct. 1, 2026

(54) TRACE ELEMENTS FORMULATION

(71) Applicant: Fresenius Kabi Deutschland GmbH, Bad Homburg v.d.H. (DE)

(72) Inventors: Marlene SCHUHMANN, Graz (AT); Claudio CRIVARO, Graz (AT); Andreas KALTENBECK, Graz (AT)

(73) Assignee: Fresenius Kabi Deutschland GmbH, Bad Homburg v.d.H. (DE)

(21) Appl. No.: 19/631,830

(22) Filed: Mar. 27, 2026

Related U.S. Application Data
(60) Provisional application No. 63/778,614, filed on Mar. 27, 2025.

(51) Int. Cl. A61K 9/00; A61J 1/06; A61K 9/08; A61K 33/04; A61K 33/16; A61K 33/18; A61K 33/26; A61K 33/30; A61K 33/32; A61K 33/34; A61K 47/12; A61K 47/26

(52) U.S. Cl. CPC A61K 9/0019; A61J 1/065; A61K 9/08; A61K 33/04; A61K 33/16; A61K 33/18; A61K 33/26; A61K 33/30; A61K 33/32; A61K 33/34; A61K 47/12; A61K 47/26

(57) ABSTRACT

The invention provides a sterile, stable injectable formulation containing a pharmaceutically acceptable salt of zinc, iron, manganese, copper, selenium, molybdenum, iodine, chromium, and/or fluorine; a chelating agent, optionally an acid and/or a base as needed for pH adjustment, and water, wherein the pH is about 3.0-4.5. The invention also provides a method for preparing the inventive formulation, a pharmaceutical product which includes a container and the inventive formulation contained therein, and a method for treating or preventing trace element deficiency in a patient by administering a therapeutically effective amount of the inventive formulation.

### CROSS REFERENCE TO RELATED APPLICATIONS

This application claims the benefit of U.S. Provisional Application No. 63/778,614, filed Mar. 27, 2025, the contents of which are incorporated herein in its entirety.

### BACKGROUND OF THE INVENTION

Trace elements play vital roles in metabolism, immune function, tissue repair, and enzyme activity, making them necessary for overall health. Deficiencies in trace elements such as zinc, copper, selenium, and chromium, can lead to various clinical manifestations depending on the element involved. Patients at high risk for trace element deficiencies include those with severe malabsorption issues, chronic diseases, major surgery, burns, or those receiving long-term parenteral nutrition or when deficiencies can arise due to inadequate dietary intake or increased losses through illness.

When patients cannot eat orally, trace elements are routinely added to intravenous nutrition solutions to prevent deficiencies. Trace element nutrition in patients refers to providing the essential needs for small amounts of minerals such as zinc, copper, selenium, manganese, chromium, and/or iodine, which are crucial for various bodily functions, particularly for patients requiring intravenous nutrition (parenteral nutrition).

Addaven™ is an injectable solution of trace elements presently marketed outside of the US for the treatment or prevention or trace element deficiencies in patients requiring parenteral nutrition. Addaven™ currently marketed outside of the US is supplied in 10 mL ampoules, each containing, per 10 mL of solution, chromic chloride hexahydrate (53.3 μg), cupric chloride dihydrate (1.02 mg), ferric chloride hexahydrate (5.40 mg), manganese chloride tetrahydrate (198 μg), potassium iodide (166 μg), sodium fluoride (2.10 mg), sodium molybdate dihydrate (48.5 μg), sodium selenite (173 μg), and zinc chloride (10.5 mg). The inactive ingredients include xylitol, hydrochloric acid, and water for injection, and the pH is about 2.5 (see, e.g., Australian Product Information—Addaven®, rev. 2024). This product is referred to herein as “conventional Addaven™.”

Conventional Addaven™ is administered intravenously (as a drip into a vein) to patients who cannot eat normally. Conventional Addaven™ is used as part of a balanced intravenous diet, together with proteins, fat, carbohydrates, salts and vitamins. The responsible physician or health care professional will normally decide the correct dose considering the patient's individual needs and potential toxicities based on the patient's condition. The recommended dose of conventional Addaven™ for adults is 10 mL per day. Patients with problems involving the liver or kidneys may receive a lower dose. Conventional Addaven™ is not recommended for use in children under 12 years of age. Conventional Addaven™ is recommended for dilution before administration with another solution, such as a compatible parenteral nutrition solution/emulsion or an aqueous diluent (see, e.g., Australian Product Information—Addaven®, rev. 2024).

There remains a need for a sterile, injectable trace element formulation with improved stability, and with improved manufacturability.

### BRIEF SUMMARY OF THE INVENTION

Conventional Addaven™ includes an overage of iodide to account for losses during manufacture and/or storage and includes xylitol, which is believed to function as a chelating agent, helping to maintain the solubility of the iron (III) compound, ferric chloride hexahydrate. The present invention is partly based on the discovery that increasing the pH of conventional Addaven™ reduced the amount of iodide loss during manufacture, but also led to ferric iron precipitation. The inventors found that increasing both the chelation strength and pH range of conventional Addaven™ reduced ferric iron precipitation and iodide loss.

The invention provides a sterile, injectable formulation containing at least one trace element selected from zinc, iron, manganese, copper, selenium, molybdenum, iodine, chromium, and fluorine or a pharmaceutically acceptable salt of the foregoing; a chelating agent, optionally an acid and/or a base as needed to adjust the pH, and water, wherein the formulation has a pH of about 3.0-4.5. The invention also provides a sterile, injectable formulation of a pharmaceutically acceptable salt containing each of the following trace elements: zinc, iron, manganese, copper, selenium, molybdenum, iodine, chromium, and fluorine; a chelating agent, optionally an acid and/or a base as needed to adjust the pH, and water, wherein the formulation has a pH of about 3.0-4.5.

In some embodiments, the formulation of the invention is a sterile, aqueous injectable solution containing from about 200 mg/L to about 20 g/L zinc sulfate heptahydrate (about 0.7 mM to about 70 mM zinc), from about 50 mg/L to about 5000 mg/L ferric chloride hexahydrate (about 0.185 mM to about 18.5 mM iron), from about 2 mg/L to about 200 mg/L manganese chloride tetrahydrate (about 0.01 mM to about 1 mM manganese), from about 10 mg/L to about 1000 mg/L copper chloride dihydrate (about 0.06 mM to about 6 mM copper), from about 2 mg/L to about 200 mg/L sodium selenite (about 0.01 mM to about 1 mM selenium), from about 0.5 mg/L to about 50 mg/L sodium molybdate dihydrate (about 0.002 mM to about 0.2 mM molybdenum), from about 2 mg/L to about 200 mg/L potassium iodide (about 0.01 mM to about 1 mM iodine), from about 0.5 mg/L to about 50 mg/L chromic chloride hexahydrate (about 0.002 mM to about 0.2 mM chromium), from about 20 mg/L to about 2000 mg/L sodium fluoride (about 0.5 to about 50 mM), a chelating agent comprising a polycarboxylic acid, a conjugate base, and a sugar alcohol, and water, wherein the formulation has a pH of about 3.0-4.5, e.g., pH 3.0-4.4, pH 3.5-4.0 or pH 3.6-4.0, e.g., pH 3.6-4.0 with a target pH of about pH 3.8. In such embodiments, the polycarboxylic acid and conjugate base may include from about 0.2 g/L to about 20 g/L trisodium citrate dihydrate (e.g., about 2134 mg/L trisodium citrate dihydrate) and from about 50 mg/L to about 5 g/L citric acid monohydrate (e.g., about 475 mg/L citric acid monohydrate), or an equivalent amount of citric acid and citrate formed in situ by adding the appropriate amount of citric acid and adjusting to pH 3.0-4.5 with a suitable base, e.g., NaOH. In such embodiments, the sugar alcohol may include from about 30 g/L to about 3000 g/L xylitol (e.g., about 300 g/L xylitol), and the water may include sterile water for injection (e.g., Water for Injection (WFI)). If needed, the pH may be adjusted with any suitable acid, e.g., hydrochloric acid (e.g., 10% aq. HCl) or sulfuric acid (e.g., 4M aq. H₂SO₄), and/or a suitable base, e.g., sodium hydroxide (e.g., 4% aq. NaOH), to achieve the desired pH. In certain embodiments, the pH is adjusted with hydrochloric acid, and no sulfuric acid is used.

The invention also includes embodiments which provide a sterile, aqueous injectable formulation, e.g., in the form of a solution, containing about 2215 mg/L zinc sulfate heptahydrate (about 7.7 mM zinc), about 540 mg/L ferric chloride hexahydrate (about 2 mM iron), about 19.8 mg/L manganese chloride tetrahydrate (about 0.1 mM manganese), about 102.3 mg/L copper chloride dihydrate (about 0.6 mM copper), about 17.3 mg/L sodium selenite (about 0.1 mM selenium), about 4.9 mg/L sodium molybdate dihydrate (about 0.02 mM molybdenum), about 16.6 mg/L potassium iodide (about 0.1 mM iodine), about 5.3 mg/L chromic chloride hexahydrate (about 0.02 mM chromium), about 210 mg/L sodium fluoride (about 5 mM fluoride), about 2134 mg/L trisodium citrate dihydrate and about 475 mg/L citric acid monohydrate (or the equivalent of amount of citric acid and citrate prepared in situ from citric acid and sodium hydroxide as described herein), about 300 g/L xylitol, and water (e.g., WFI), wherein the formulation has a pH of about 3.0-4.5, e.g., about pH 3.0-4.4, about pH 3.5-4.0 or about pH 3.6-4.0, e.g., about pH 3.6-4.0 with a target pH of about pH 3.8.

The formulation of the invention is remarkably stable/resistant to iodide loss during manufacture, after autoclaving, and during storage. For example, the formulation of the invention includes embodiments in which the iodide concentration in the formulation decreases by no more than about 5% after autoclaving, e.g., at about 115° C. for about 30-40 minutes, and/or after storing at about 25° C. for about 3-24 months, at about 30° C. for about 3-12 months, and/or at about 40° C. for about 1-6 months. The formulation of the invention thus avoids the need to add any overage of iodide beyond the labeled content and allows for a tighter product specification range of 90-110% (±10%) of the labeled content as compared to conventional Addaven™, which may require adding a 20% overage of iodide during manufacture and/or may have a wider product specification of ±20% of the labeled content, e.g., to account for iodide loss during storage. The formulation of the invention also is stable/resistant to fluoride loss during manufacture, after autoclaving, and during storage. For example, the formulation of the invention includes embodiments in which the fluoride concentration in the formulation decreases by no more than about 5% after autoclaving, e.g., at about 115° C. for about 30-40 minutes, and/or after storing at about 25° C. for about 3-24 months, at about 30° C. for about 3-12 months, and/or at about 40° C. for about 1-6 months.

The formulation of the invention is also remarkably stable/resistant to changes in coloration during manufacture, after autoclaving, and during storage. For example, the formulation of the invention includes embodiments in which the formulation is no more intensively colored than reference solution Greenish Yellow 1 (GY1) as determined by the European Pharmacopoeia Chapter 2.2.2 (Ph.Eur.2.2.2) or U.S. Pharmacopeia Chapter 1061 (USP<1061>) after storing at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

The formulation of the invention is also stable/resistant to changes in pH during manufacture, after autoclaving, and during storage. For example, the formulation of the invention includes embodiments in which the pH of the formulation changes by no more than about 0.5 pH units, e.g., no more than about 0.4 pH units, no more than about 0.3 pH units, no more than about 0.2 pH units, or no more than about 0.1 pH units, after autoclaving, e.g., at about 115° C. for about 30-40 minutes, and/or after storing at about 25° C. for about 6 months.

The formulation of the invention also is essentially non-corrosive to stainless steel, and thus compatible with stainless steel manufacturing equipment, allowing for the production of the formulation in stainless steel tanks with minimal risks to both product and patients due to corrosion and/or leaching of metals or coating materials (e.g., Teflon).

The formulation of the invention is also remarkably stable/resistant to formation of visible and subvisible particulates matter during manufacture, after autoclaving, and during storage. For example, the formulation of the invention includes embodiments in which the formulation is clear after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. The formulation of the invention includes embodiments in which the formulation is essentially free of visible particles as determined by USP<790> and includes, per container, no more than 6000 subvisible particles greater than or equal to 10 m, and no more than 600 subvisible particles greater than or equal to 25 μm as determined by USP<788> after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

The invention also provides a method for preparing the formulation of the invention, which method includes, for example, dissolving in water from about 200 mg/L to about 20 g/L zinc sulfate heptahydrate, from about 50 mg/L to about 5000 mg/L ferric chloride hexahydrate, from about 2 mg/L to about 200 mg/L manganese chloride tetrahydrate, from about 10 mg/L to about 1000 mg/L copper chloride dihydrate, from about 2 mg/L to about 200 mg/L sodium selenite, from about 0.5 mg/L to about 50 mg/L sodium molybdate dihydrate, from about 2 mg/L to about 200 mg/L potassium iodide, from about 0.5 mg/L to about 50 mg/L chromic chloride hexahydrate, from about 20 mg/L to about 2000 mg/L sodium fluoride, a chelating agent, e.g., citric acid, citrate, and xylitol as described herein, wherein the formulation has a pH of about 3.0-4.5, e.g., pH 3.0-4.4, pH 3.5-4.0 or pH 3.6-4.0, e.g., pH 3.6-4.0 with a target pH of about pH 3.8. The method also may include optionally dissolving an acid and/or a base if needed to adjust the pH to about pH 3.0-4.5, e.g., pH 3.0-4.4, pH 3.5-4.0 or pH 3.6-4.0, e.g., pH 3.6-4.0 with a target pH of about pH 3.8, as described herein, and optionally sterilizing the formulation as described herein, e.g., by autoclaving at about 115° C. for about 30-40 minutes.

The invention additionally provides a pharmaceutical product which includes a container and the formulation of the invention (including the formulation prepared by the method of the invention) contained therein. The container may be sealed with any seal/closure suitable for intravenous or parenteral pharmaceutical solutions. The container may include a primary container, e.g., as a single container, or may include a primary container combined with one or more secondary containers, e.g., which may serve to protect, surround, and/or contain the primary container. The primary container also may include a syringe, wherein the syringe barrel may include or be made of, e.g., glass or plastic. In some embodiments, the container used for the product of the invention is a vial, ampoule, syringe, or bag. The product of the invention thus includes embodiments in which the container is a vial such as, for example, a plastic vial, e.g., a 10 mL polypropylene vial or a 100 mL polypropylene vial, or a glass vial.

The invention further provides a method for treating or preventing trace element deficiency in a patient, which method includes administering to the patient a therapeutically effective amount of the formulation of the invention, including the formulation prepared by the method of the invention, and the formulation contained in the product of the invention. The formulation of the invention may be administered by any suitable method, including methods prescribed for the administration of conventional Addaven™, for example, intravenous injection or infusion. The formulation of the invention also may be administered to treat any indication for which conventional Addaven™ may be approved. The formulation of the invention may be administered to the patient directly without prior dilution although, preferably, the formulation may be diluted before administration to the patient.

### DETAILED DESCRIPTION OF THE INVENTION

The invention provides a sterile, injectable formulation, e.g., solution, composition, mixture, emulsion, or the like, containing at least one (e.g., one, two, three, four, five, six, seven, eight, or nine) trace element selected from zinc, iron, manganese, copper, selenium, molybdenum, iodine, chromium, and fluorine or a pharmaceutically acceptable salt of the foregoing; a chelating agent, optionally an acid and/or a base as needed to adjust the pH, and water, wherein the formulation has a pH of about 3.0-4.5. In some embodiments, the formulation of the invention includes a sterile, injectable solution containing a pharmaceutically acceptable salt of each of the following trace elements: zinc, iron, manganese, copper, selenium, molybdenum, iodine, chromium, and fluorine; a chelating agent, optionally an acid and/or a base as needed to adjust the pH, and water, wherein the formulation has a pH of about 3.0-4.5.

In certain embodiments, the formulation of the invention is free of, or substantially free of, one or more trace element. For example, a formulation of the invention may be free of, or substantially free of, iron, fluorine, molybdenum, and/or chromium, or a pharmaceutically acceptable salt the foregoing. By “substantially free of” a trace element is meant that no amount of the trace element is added during manufacture of the formulation, and any amount present is an impurity or contaminant derived from, e.g., another component of the formulation (e.g., water or an included trace element), primary container, and/or manufacturing equipment. By “free of” a trace element is meant that no amount of the trace element, for example, as detected by standard analytical techniques, is added during the manufacture of the formulation and no amount of the trace element, for example, as detected by standard analytical techniques, is present as an impurity or contaminant derived from, e.g., another component of the formulation, primary container, and/or manufacturing equipment.

In a preferred embodiment, the formulation of the invention is free of fluorine and chromium or free of fluorine, chromium, and iron. In another embodiment, the formulation of the invention is substantially free of fluorine and chromium or substantially free of fluorine, chromium, and iron. Accordingly, in one embodiment, a formulation of the invention comprises, consists of, or consists essentially of the following trace elements: zinc, manganese, copper, selenium, iodine, and molybdenum. In another embodiment, a formulation of the invention comprises, consists of, or consists essentially of the following trace elements: zinc, manganese, copper, selenium, and iodine.

The pharmaceutically acceptable salts of the one or more trace elements may include any salt(s) suitable for intravenous administration to patients who require one or more of such trace elements as part of a balanced diet, but are unable to obtain the nutritional requirements orally. The pharmaceutically acceptable salt of zinc may include a non-halogen salt of zinc such as, for example, zinc sulfate, e.g., zinc sulfate heptahydrate. The pharmaceutically acceptable salt of iron may include, for example, ferric chloride, e.g., ferric chloride hexahydrate. The pharmaceutically acceptable salt of manganese may include, for example, manganese chloride, e.g., manganese chloride tetrahydrate. The pharmaceutically acceptable salt of copper may include, for example, copper (II) chloride, e.g., copper (II) chloride dihydrate (cupric chloride dihydrate). The pharmaceutically acceptable salt of selenium may include, for example, an alkali metal salt of selenium such as, e.g., sodium selenite, or the selenium may be provided as selenious acid. The pharmaceutically acceptable salt of molybdenum may include an alkali metal salt of molybdenum such as, for example, sodium molybdate, e.g., sodium molybdate dihydrate. The pharmaceutically acceptable salt of iodine may include, for example, an alkali metal salt of iodine, e.g., potassium iodide. The pharmaceutically acceptable salt of chromium may include, for example, chromic chloride, e.g., chromic chloride hexahydrate. The pharmaceutically acceptable salt of fluorine may include, for example, an alkali metal salt of fluorine such as, e.g., sodium fluoride.

The chelating agent present in a formulation of the invention may include any compound capable of sequestering metal ions, e.g., in solution, that is suitable for intravenous or parenteral administration. Suitable chelating agents include, without limitation, a sugar alcohol (e.g., xylitol, mannitol, sorbitol), a polycarboxylic acid and/or conjugate base thereof (e.g., citric acid, citrate, oxalic acid, oxalate, tartaric acid, tartrate, malic acid, malate), a monocarboxylic acid and/or conjugate base thereof (e.g., acetic acid, acetate, lactic acid, lactate), a sugar acid and/or salt thereof (e.g., gluconic acid, gluconate, aldonic acid, aldonate, lactobionic acid, lactobionate), an amino acid (e.g., cysteine, glutamic acid, histidine, aspartic acid), ethylenediaminetetraacetic acid (EDTA), ethylene glycol-bis(O-aminoethyl ether)-N,N,N′,N′-tetraacetic acid (EGTA), diethylenetriaminepentaacetic acid (DTPA), N-(hydroxyethyl)ethylenediaminetriacetic acid (HEDTA), aminotriacetic acid, 2,3-dimercapto-1-propanesulfonic acid, dimercaptosuccinic acid, ethylenediamine, dimercaprol, and salts, hydrates, and/or derivatives thereof.

A formulation of the invention also may include two or more, e.g., three or more, four or more, or five or more, chelating agents. In some embodiments, the chelating agent comprises a sugar alcohol, a polycarboxylic acid, and a conjugate base. In certain embodiments, the chelating agent comprises xylitol, citric acid, and citrate. The amount of each chelating agent present in a formulation of the invention will depend upon several factors, including pH, chelation strength, solubility, and presence of additional chelating agents, and may be determined by a person of ordinary skill in the art. In some embodiments, the chelating agent comprises xylitol present in a concentration of 10 mg/mL to 1000 mg/mL, e.g., 50 mg/mL to 800 mg/mL, 100 mg/mL to 500 mL, 200 mg/mL to 400 mL. In certain embodiments, the chelating agent comprises xylitol present at a concentration of about 100 mg/mL, about 200 mg/mL, about 250 mg/mL, about 300 mg/mL, about 350 mg/mL, about 400 mg/mL, or about 500 mg/mL.

In some embodiments, the chelating agent comprises citrate provided as, for example, a combination of citric acid and a salt of citric acid. In some embodiments, the citrate is provided as a combination of citric acid and an alkali metal salt of citric acid, e.g., combinations of citric acid (e.g., citric acid monohydrate) and a sodium salt of citric acid (e.g., trisodium citrate dihydrate).

In certain embodiments, the chelating agent comprises a combination of citric acid and sodium citrate obtained, for example, by dissolving a suitable form of sodium citrate such as, for example, trisodium citrate dihydrate, and a suitable form of citric acid such as, for example, citric acid monohydrate, in the appropriate ratio needed to achieve a pH of about 3.0-4.5, and, if needed, adjusting the pH with a suitable acid (e.g., HCl or H₂SO₄) or base (e.g., NaOH), to achieve the desired pH. In other embodiments, the chelating agent is obtained by dissolving a suitable form of citric acid such as, for example, citric acid monohydrate, in the amount needed to produce in situ an equivalent amount of citrate after adjusting the pH to about 3.0-4.5 with a suitable base (e.g., NaOH). Preferably, the chelating agent may be prepared by combining trisodium citrate dihydrate and citric acid monohydrate in the appropriate ratio needed to achieve a pH of about 3.0-4.5, and, if needed, by adjusting the pH with a suitable acid (e.g., HCl or H₂SO₄) or base (e.g., NaOH) to achieve the desired pH.

Any grade of water suitable for use in the manufacture of injectable or parenteral pharmaceutical formulations may be used as an aqueous solvent for the formulation of the invention. Suitable grades of water may include, for example, sterile, non-pyrogenic grades of water used in the preparation of injectable or parenteral formulations, e.g., Water for Injection (WFI).

The concentrations of the one or more trace elements may include any concentration suitable for intravenous administration in an aqueous formulation alone, e.g., without prior dilution, of after dilution with a suitable diluent, which may contain, e.g., one or more proteins, fats, carbohydrates, other nutritional salts, and vitamins, to patients who require one or more of such trace elements as part of a balanced diet, but who are unable to obtain the nutritional requirements orally.

The pH of the formulation of the invention preferably ranges from about pH 3.0 to about pH 4.5. In some embodiments, the pH of the formulation ranges from about pH 3.0 to about pH 4.4, e.g, pH 3.1-4.4, pH 3.2-4.4, pH 3.3-4.4, pH 3.4-4.4, pH 3.5-4.4, pH 3.6-4.4, pH 3.7-4.4, pH 3.8-4.4, pH 3.1-4.3, pH 3.1-4.2, pH 3.1-4.1, pH 3.1-4.0, pH 3.1-3.9, pH 3.1-3.8, pH 3.2-4.4, pH 3.2-4.3, pH 3.2-4.2, pH 3.2-4.1, pH 3.2-4.1, pH 3.2-4.0, pH 3.2-3.9, pH 3.2-3.8, pH 3.3-4.3, pH 3.3-4.2, pH 3.3-4.1, pH 3.3-4.0, pH 3.3-3.9, pH 3.3-3.8, pH 3.4-4.3, pH 3.4-4.2, pH 3.4-4.1, pH 3.4-4.0, pH 3.4-3.9, pH 3.4-3.8, pH 3.5-4.3, pH 3.5-4.2, pH 3.5-4.1, pH 3.5-4.0, pH 3.5-3.9, pH 3.5-3.8, pH 3.6-4.3, pH 3.6-4.2, pH 3.6-4.1, pH 3.6-4.0, pH 3.6-3.9, pH 3.6-3.8, pH 3.7-4.3, pH 3.7-4.2, pH 3.7-4.1, pH 3.7-4.0, or pH 3.7-3.9. In some embodiments, the pH of the formulation ranges from about pH 3.5 to about pH 4.0. In some embodiments, the pH of the formulation ranges from about pH 3.6 to about pH 4.0, e.g., from about pH 3.6 to about pH 4.0 with a target in-process pH of about pH 3.8.

If needed, the pH of the formulation may be adjusted with any suitable acid such as, for example, HCl (e.g., 10% aq. HCl) or H₂SO₄ (e.g., 4M aq. H₂SO₄), and/or a suitable base such as, for example, NaOH (e.g., 4% aq. NaOH), to achieve the desired pH. Unless otherwise indicated, embodiments of the of the invention as described herein may optionally include a suitable acid and/or base as needed to achieve a desired pH. In certain embodiments, the pH is adjusted with hydrochloric acid, and no sulfuric acid is used.

The formulation of the invention may further include at least one additional excipient. Non limiting examples of suitable excipients may include, for example, buffers, tonicity agents, antioxidants, preservatives, diluents, salts, stabilizers, solubilizers, and the like, and combinations thereof.

In some embodiments, the formulation of the invention is a sterile, aqueous injectable solution containing from about 200 mg/L to about 20 g/L zinc sulfate heptahydrate, from about 50 mg/L to about 5000 mg/L ferric chloride hexahydrate, from about 2 mg/L to about 200 mg/L manganese chloride tetrahydrate, from about 10 mg/L to about 1000 mg/L copper chloride dihydrate, from about 2 mg/L to about 200 mg/L sodium selenite, from about 0.5 mg/L to about 50 mg/L sodium molybdate dihydrate, from about 2 mg/L to about 200 mg/L potassium iodide, from about 0.5 mg/L to about 50 mg/L chromic chloride hexahydrate, from about 20 mg/L to about 2000 mg/L sodium fluoride, a chelating agent comprising a polycarboxylic acid, a conjugate base, and a sugar alcohol, and water, wherein the formulation has a pH of about 3.0-4.5, e.g., pH 3.0-4.4, pH 3.5-4.0 or pH 3.6-4.0, e.g., pH 3.6-4.0 with a target pH of about pH 3.8. In such embodiments, the polycarboxylic and conjugate base may include from about 0.2 g/L to about 20 g/L trisodium citrate dihydrate (e.g., about 2134 mg/L trisodium citrate dihydrate) and from about 50 mg/L to about 5 g/L citric acid monohydrate (e.g., about 475 mg/L citric acid monohydrate), or an equivalent amount of citric acid and citrate formed in situ by adding the appropriate amount of citric acid (e.g., about 2.0 g/L of citric acid monohydrate) and adjusting the pH with a suitable base, e.g., NaOH, to achieve the desired pH. In such embodiments, the sugar alcohol may include, e.g., from about 30 g/L to about 3000 g/L xylitol (e.g., about 300 g/L xylitol), and the water may include, e.g., sterile water for injection (e.g., WFI).

In some embodiments, the formulation of the invention is a sterile, aqueous injectable solution comprising, consisting essentially of, or consisting of, from about 1 mg/L to about 200 mg/L selenium. For example, the amount of selenium present in a formulation of the invention can be about any of the following values: 1 mg/L, 2 mg/L, 3 mg/L, 3.2 mg/L, 3.5 mg/L, 3.8 mg/L, 4 mg/L, 5 mg/L, 6 mg/L, 7 mg/L, 8 mg/L, 9 mg/L, 10 mg/L, 20 mg/L, 30 mg/L, 40 mg/L, 50 mg/L, 60 mg/L, 70 mg/L, 80 mg/L, 90 mg/L, 91 mg/L, 92 mg/L, 93 mg/L, 94 mg/L, 95 mg/L, 96 mg/L, 97 mg/L, 98 mg/L, 99 mg/L, 100 mg/L, 110 mg/L, 120 mg/L, 130 mg/L, 140 mg/L, 150 mg/L, or 200 mg/L, or within a range bounded by any two of the foregoing values (e.g., 2-200 mg/L, 3-100 mg/L, 2-3.8 mg/L, 91-200 mg/L, or 95-150 mg/L). In certain embodiments, the selenium is provided as sodium selenite. In other embodiments, the selenium is provided as selenious acid. A selenium formulation of the invention preferably can have the same stability characteristics (e.g., pH, assay, aluminum content, particulates, color, clarity) as described herein for formulations of the invention comprising two or more trace elements.

In some embodiments, the formulation of the invention is a sterile, aqueous injectable solution containing about 2215 mg/L zinc sulfate heptahydrate, about 540 mg/L ferric chloride hexahydrate, about 19.8 mg/L manganese chloride tetrahydrate, about 102.3 mg/L copper chloride dihydrate, about 17.3 mg/L sodium selenite, about 4.9 mg/L sodium molybdate dihydrate, about 16.6 mg/L potassium iodide, about 5.3 mg/L chromic chloride hexahydrate, about 210 mg/L sodium fluoride, a chelating agent comprising a polycarboxylic acid, a conjugate base, and a sugar alcohol, and water, wherein the formulation has a pH of about 3.0-4.5, e.g., about pH 3.0-4.4, about pH 3.5-4.0 or about pH 3.6-4.0, e.g., about pH 3.6-4.0 with a target pH of about pH 3.8. In such embodiments, the polycarboxylic acid and conjugate base may include from about 0.2 g/L to about 20 g/L trisodium citrate dihydrate (e.g., about 2134 mg/L trisodium citrate dihydrate) and from about 50 mg/L to about 5 g/L citric acid monohydrate (e.g., about 475 mg/L citric acid monohydrate), or an equivalent amount of citric acid and citrate formed in situ by adding the appropriate amount of citric acid monohydrate (e.g., about 2.0 g/L) and adjusting the pH with a suitable base, e.g., NaOH, to achieve the desired pH. Also in such embodiments, the sugar alcohol may include, e.g., from about 30 g/L to about 3000 g/L xylitol (e.g., about 300 g/L xylitol), and the water may include, e.g., sterile water for injection (e.g., WFI).

In some embodiments, the formulation of the invention is a sterile, aqueous injectable solution containing about 2215 mg/L zinc sulfate heptahydrate, about 540 mg/L ferric chloride hexahydrate, about 19.8 mg/L manganese chloride tetrahydrate, about 102.3 mg/L copper chloride dihydrate, about 17.3 mg/L sodium selenite, about 4.9 mg/L sodium molybdate dihydrate, about 16.6 mg/L potassium iodide, about 5.3 mg/L chromic chloride hexahydrate, about 210 mg/L sodium fluoride, a chelating agent comprising about 2134 mg/L trisodium citrate dihydrate and about 475 mg/L citric acid monohydrate (or the equivalent of such chelator prepared in situ from citric acid (e.g., about 2.0 g/L citric acid monohydrate) and sodium hydroxide as described herein), and about 300 g/L xylitol, and water, wherein the formulation has a pH of about 3.0-4.5, e.g., about pH 3.0-4.4, about pH 3.5-4.0 or about pH 3.6-4.0, e.g., about pH 3.6-4.0 with a target pH of about pH 3.8. In such embodiments, the water may include sterile water for injection (e.g., WFI), and the pH is preferably about 3.6-4.0, e.g., about pH 3.6-4.0 with a target pH of about pH 3.8.

In some embodiments, the formulation of the invention is a sterile, injectable solution containing a pharmaceutically acceptable salt of zinc, a chelating agent, optionally an acid and/or a base as needed to adjust the pH, and water, wherein the formulation has a pH of about 3.0-4.5. In certain embodiments, the formulation comprises from about 200 mg/L to about 20 g/L zinc sulfate heptahydrate, a chelating agent comprising a polycarboxylic acid, a conjugate base, and a sugar alcohol, and water, wherein the formulation has a pH of about 3.0-4.5, e.g., pH 3.0-4.4, pH 3.5-4.0 or pH 3.6-4.0, e.g., pH 3.6-4.0 with a target pH of about pH 3.8.

In other embodiments, the formulation of the invention is a sterile, injectable solution containing a pharmaceutically acceptable salt of iron, a chelating agent, optionally an acid and/or a base as needed to adjust the pH, and water, wherein the formulation has a pH of about 3.0-4.5. In certain embodiments, the formulation comprises from about 50 mg/L to about 5000 mg/L ferric chloride hexahydrate, a chelating agent comprising a polycarboxylic acid, a conjugate base, and a sugar alcohol, and water, wherein the formulation has a pH of about 3.0-4.5, e.g., pH 3.0-4.4, pH 3.5-4.0 or pH 3.6-4.0, e.g., pH 3.6-4.0 with a target pH of about pH 3.8.

In some embodiments, the formulation of the invention is a sterile, injectable solution containing a pharmaceutically acceptable salt of manganese, a chelating agent, optionally an acid and/or a base as needed to adjust the pH, and water, wherein the formulation has a pH of about 3.0-4.5. In certain embodiments, the formulation comprises from about 2 mg/L to about 200 mg/L manganese chloride tetrahydrate, a chelating agent comprising a polycarboxylic acid, a conjugate base, and a sugar alcohol, and water, wherein the formulation has a pH of about 3.0-4.5, e.g., pH 3.0-4.4, pH 3.5-4.0 or pH 3.6-4.0, e.g., pH 3.6-4.0 with a target pH of about pH 3.8.

In other embodiments, the formulation of the invention is a sterile, injectable solution containing a pharmaceutically acceptable salt of copper, a chelating agent, optionally an acid and/or a base as needed to adjust the pH, and water, wherein the formulation has a pH of about 3.0-4.5. In certain embodiments, the formulation comprises from about 10 mg/L to about 1000 mg/L copper chloride dihydrate, a chelating agent comprising a polycarboxylic acid, a conjugate base, and a sugar alcohol, and water, wherein the formulation has a pH of about 3.0-4.5, e.g., pH 3.0-4.4, pH 3.5-4.0 or pH 3.6-4.0, e.g., pH 3.6-4.0 with a target pH of about pH 3.8.

In some embodiments, the formulation of the invention is a sterile, injectable solution containing a pharmaceutically acceptable salt of selenium (or selenious acid), a chelating agent, optionally an acid and/or a base as needed to adjust the pH, and water, wherein the formulation has a pH of about 3.0-4.5. In certain embodiments, the formulation comprises from about 2 mg/L to about 200 mg/L sodium selenite, a chelating agent comprising a polycarboxylic acid, a conjugate base, and a sugar alcohol, and water, wherein the formulation has a pH of about 3.0-4.5, e.g., pH 3.0-4.4, pH 3.5-4.0 or pH 3.6-4.0, e.g., pH 3.6-4.0 with a target pH of about pH 3.8.

In other embodiments, the formulation of the invention is a sterile, injectable solution containing a pharmaceutically acceptable salt of molybdenum, a chelating agent, optionally an acid and/or a base as needed to adjust the pH, and water, wherein the formulation has a pH of about 3.0-4.5. In certain embodiments, the formulation comprises from about 0.5 mg/L to about 50 mg/L sodium molybdate dihydrate, a chelating agent comprising a polycarboxylic acid, a conjugate base, and a sugar alcohol, and water, wherein the formulation has a pH of about 3.0-4.5, e.g., pH 3.0-4.4, pH 3.5-4.0 or pH 3.6-4.0, e.g., pH 3.6-4.0 with a target pH of about pH 3.8.

In some embodiments, the formulation of the invention is a sterile, injectable solution containing a pharmaceutically acceptable salt of iodine, a chelating agent, optionally an acid and/or a base as needed to adjust the pH, and water, wherein the formulation has a pH of about 3.0-4.5. In certain embodiments, the formulation comprises from about 2 mg/L to about 200 mg/L potassium iodide, a chelating agent comprising a polycarboxylic acid, a conjugate base, and a sugar alcohol, and water, wherein the formulation has a pH of about 3.0-4.5, e.g., pH 3.0-4.4, pH 3.5-4.0 or pH 3.6-4.0, e.g., pH 3.6-4.0 with a target pH of about pH 3.8.

In other embodiments, the formulation of the invention is a sterile, injectable solution containing a pharmaceutically acceptable salt of chromium, a chelating agent, optionally an acid and/or a base as needed to adjust the pH, and water, wherein the formulation has a pH of about 3.0-4.5. In certain embodiments, the formulation comprises from about 0.5 mg/L to about 50 mg/L chromic chloride hexahydrate, a chelating agent comprising a polycarboxylic acid, a conjugate base, and a sugar alcohol, and water, wherein the formulation has a pH of about 3.0-4.5, e.g., pH 3.0-4.4, pH 3.5-4.0 or pH 3.6-4.0, e.g., pH 3.6-4.0 with a target pH of about pH 3.8.

In some embodiments, the formulation of the invention is a sterile, injectable solution containing a pharmaceutically acceptable salt of fluorine, a chelating agent, optionally an acid and/or a base as needed to adjust the pH, and water, wherein the formulation has a pH of about 3.0-4.5. In certain embodiments, the formulation comprises from about 20 mg/L to about 2000 mg/L sodium fluoride, a chelating agent comprising a polycarboxylic acid, a conjugate base, and a sugar alcohol, and water, wherein the formulation has a pH of about 3.0-4.5, e.g., pH 3.0-4.4, pH 3.5-4.0 or pH 3.6-4.0, e.g., pH 3.6-4.0 with a target pH of about pH 3.8.

The invention also is predicated, at least in part, on the surprising and unexpected discovery that substantial improvements in stability, as well as substantial improvements in the ease of commercial scale manufacture, may be achieved with the formulation of the invention relative to other trace elements formulations such as conventional Addaven™. For example, the formulation of the invention was found to be surprisingly and unexpectedly more stable than conventional Addaven™ in terms of iodide loss. The formulation of the invention thus avoids the need to add any overage of iodide beyond the labeled content, and allows for a tighter product specification (e.g., 90-110% (±10%) of the labeled content) as compared to conventional Addaven™, which may require adding a 20% overage of iodide and may have a wider product specification of ±20% of the labeled content, e.g., to account for iodide loss. The formulation of the invention also was found to be surprisingly and unexpectedly stable in terms of fluoride loss during manufacture, after autoclaving, and during long-term storage particularly at lower pH values. The formulation of the invention additionally was found to be surprisingly and unexpectedly stabile/resistant to changes in coloration, changes in pH, and formation of particulates during manufacture, after autoclaving, and during long-term storage. The formulation of the invention further was surprisingly and unexpectedly found to be essentially non-corrosive to stainless steel, unlike the conventional Addaven™ formulation which is corrosive to stainless steel. The formulation of the invention may thus be manufactured in stainless steel tanks with minimal risks to both product and patients due to corrosion.

In some embodiments, the iodide concentration in the formulation of the invention decreases by no more than about 5% after autoclaving, e.g., at about 115° C. for about 30-40 minutes. For example, the iodide concentration in the formulation of the invention may decrease by no more than about 4% after autoclaving, or may decrease by no more than about 3% after autoclaving, or may decrease by no more than about 2% after autoclaving, or may decrease by no more than about 1% after autoclaving.

The invention also includes embodiments in which the iodide concentration in the formulation decreases by no more than about 5% after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. For example, the iodide concentration in the formulation of the invention may decrease by no more than about 4%, or may decrease by no more than about 3%, or may decrease by no more than about 2%, or may decrease by no more than about 1%, after storage at about 25° C. for about 3-24 months, at about 30° C. for about 3-12 months, and/or at about 40° C. for about 1-6 months without autoclaving, or after autoclaving at about 115° C., e.g., for about 30-40 minutes and then storing at about 25° C. for about 3-24 months, at about 30° C. for about 3-12 months, and/or at about 40° C. for about 1-6 months.

The formulation of the invention thus avoids the need to include any overage of iodide beyond the labeled content, and allows for a tighter product specification range of 90-110% (±10%) for the labeled content, whereas the conventional Addaven™ formulation may require adding a 20% overage of iodide and/or a wider product specification of ±20% of the labeled/added content, e.g., to account for iodide loss.

In some embodiments, the fluoride concentration in the formulation of the invention decreases by no more than about 5% after autoclaving, e.g., at about 115° C. for about 30-40 minutes. For example, the fluoride concentration in the formulation of the invention may decrease by no more than about 4% after autoclaving, or may decrease by no more than about 3% after autoclaving, or may decrease by no more than about 2% after autoclaving, or may decrease by no more than about 1% after autoclaving.

The invention also includes embodiments in which the fluoride concentration in the formulation of the invention decreases by no more than about 5% after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. For example, the fluoride concentration in the formulation of the invention may decrease by no more than about 4%, or may decrease by no more than about 3%, or may decrease by no more than about 2%, or may decrease by no more than about 1% after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

The content of each trace element in a formulation of the invention may be determined by any suitable method, including but not limited to, inductively coupled plasma mass spectrometry (ICP-MS), inductively coupled plasma optical emission spectroscopy (ICP-OES), high-performance liquid chromatography (HPLC), or ion-selective electrode (ISE). In certain embodiments, iodide content is determined by ICP-MS or HPLC, fluoride content is determined by ISE, and the contents of any remaining trace elements (other than iodide and fluoride) are determined by ICP-MS or ICP-OES using standard methodologies.

In some embodiments, the formulation of the invention may contain, or may fall within a product specification requiring, 90%-110% of a labeled amount of each of the at least one trace elements present in the formulation after storage of the product at about 25° C. for about 3-24 months, at about 30° C. for about 3-12 months, and/or at about 40° C. for about 1-6 months.

In some embodiments, the formulation of the invention is not more intensively colored than reference solution Greenish Yellow 1 (GY1) as determined by Ph.Eur.2.2.2 or USP<1061> after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

In some embodiments, the pH of the formulation of the invention changes by no more than about 0.5 pH units after autoclaving, e.g., at about 115° C. for about 30-40 minutes. For example, the pH of the formulation of the invention may change by no more than about 0.4 pH units after autoclaving, or may change by no more than about 0.3 pH units after autoclaving, or may change by no more than about 0.2 pH units after autoclaving, or may change by no more than about 0.1 pH units after autoclaving.

In some embodiments, the pH of the formulation of the invention changes by no more than about 0.5 pH units after storing the formulation at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. For example, the pH of the formulation of the invention may change by no more than about 0.4 pH units, or may change by no more than about 0.3 pH units, or may change by no more than about 0.2 pH units, or may change by no more than about 0.1 pH units, after storing the formulation at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

In some embodiments, the formulation of the invention is essentially non-corrosive to stainless steel. For example, the formulation of the invention may contain no more than about 0.1 mg/L of nickel after incubating the formulation in the presence of stainless steel for about 4 weeks at room temperature. The formulation of the invention is thus compatible with stainless steel manufacturing equipment, allowing for the production of the formulation in stainless steel tanks with minimal risks to both product and patients due to corrosion.

The invention also includes embodiments in which the formulation of the invention is clear after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

In some embodiments, the formulation of the invention is essentially free of visible particles as determined by USP<790> and includes, per container, not more than 6000 subvisible particles greater than or equal to 10 μm, and not more than 600 subvisible particles greater than or equal to 25 μm as determined by USP<788> after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. In such embodiments, the formulation of the invention may be essentially free from visible particles as determined by USP<790>, and may contain, per container, not more than 600, e.g., not more than 500, not more than 400, not more than 300, not more than 200, not more than 100, not more than 50, not more than 40, or not more than 30, subvisible particles greater than or equal to 10 μm, and not more than 60, e.g., not more than 50, not more than 40, not more than 30, not more than 20, or not more than 10, subvisible particles greater than or equal to 25 μm as determined by USP<788>, after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. In such embodiments, the formulation of the invention may remain clear after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, then storing at about 25° C. for about 3-24 months, at about 30° C. for about 3-12 months, and/or at about 40° C. for about 1-6 months.

In some embodiments, the formulation of the invention contains not more than about 250 mcg/L aluminum, e.g., not more than about 200 mcg/L aluminum, not more than about 150 mcg/L aluminum, not more than about 100 mcg/L aluminum, not more than about 75 mcg/L aluminum, or not more than about 50 mcg/L aluminum, as determined, for example, by graphite furnace atomic absorption spectrometry (GFAAS) or inductively coupled plasma-sector field mass spectrometry (ICP-SFMS), after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. In certain embodiments, the amount of aluminum present in formulation of the invention after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months, is beneath the level of detection by the selected analytical method, e.g., 20 mcg/L.

The invention also provides a method for preparing the formulation of the invention, which method includes, for example, dissolving in water from about 200 mg/L to about 20 g/L zinc sulfate heptahydrate, from about 50 mg/L to about 5000 mg/L ferric chloride hexahydrate, from about 2 mg/L to about 200 mg/L manganese chloride tetrahydrate, from about 10 mg/L to about 1000 mg/L copper chloride dihydrate, from about 2 mg/L to about 200 mg/L sodium selenite, from about 0.5 mg/L to about 50 mg/L sodium molybdate dihydrate, from about 2 mg/L to about 200 mg/L potassium iodide, from about 0.5 mg/L to about 50 mg/L chromic chloride hexahydrate, from about 20 mg/L to about 2000 mg/L sodium fluoride, a chelating agent, e.g., a polycarboxylic acid, a conjugate base, and a sugar alcohol as described herein, wherein the formulation has a pH of about 3.0-4.5, e.g., pH 3.0-4.4, pH 3.5-4.0 or pH 3.6-4.0, e.g., pH 3.6-4.0 with a target pH of about pH 3.8. The method also may include optionally dissolving an acid and/or a base if needed to adjust the pH to about pH 3.0-4.5, e.g., pH 3.0-4.4, pH 3.5-4.0 or pH 3.6-4.0, e.g., pH 3.6-4.0 with a target pH of about pH 3.8.

The method of the invention may include adding and/or preparing in situ any suitable polycarboxylic acid and conjugate base chelating agent as described herein. For example, the method may include preparing a chelating agent as described herein, e.g., by dissolving from about 0.2 g/L to about 20 g/L trisodium citrate dihydrate (e.g., about 2134 mg/L trisodium citrate dihydrate) and from about 50 mg/L to about 5 g/L citric acid monohydrate (e.g., about 475 mg/L citric acid monohydrate) in the water. Alternatively, an equivalent chelating agent may be formed in situ as described herein, e.g., by dissolving the appropriate amount of citric acid (e.g., about 2.0 g/L citric acid monohydrate) in the water, and adjusting the pH with a suitable base, e.g., NaOH, to achieve the desired pH.

The method of the invention also may include adding another suitable chelating agent such as, for example, a sugar alcohol, in the water as described herein. For example, the method may include dissolving, e.g., from about 30 g/L to about 3000 g/L xylitol or, e.g., about 300 g/L xylitol, in the water as described herein.

In some embodiments, the method of invention includes dissolving in water about 2215 mg/L zinc sulfate heptahydrate, about 540 mg/L ferric chloride hexahydrate, about 19.8 mg/L manganese chloride tetrahydrate, about 102.3 mg/L copper chloride dihydrate, about 17.3 mg/L sodium selenite, about 4.9 mg/L sodium molybdate dihydrate, about 16.6 mg/L potassium iodide, about 5.3 mg/L chromic chloride hexahydrate, and about 210 mg/L sodium fluoride. In such embodiments, the chelating agent may include any suitable chelating agent as described herein such as, for example, from about 0.2 g/L to about 20 g/L trisodium citrate dihydrate (e.g., about 2134 mg/L trisodium citrate dihydrate) and from about 50 mg/L to about 5 g/L citric acid monohydrate (e.g., about 475 mg/L citric acid monohydrate). For example, the chelating agent preparation may include dissolving about 2134 mg/L trisodium citrate dihydrate and about 475 mg/L citric acid monohydrate in the water. Alternatively, an equivalent chelating agent may be formed in situ by dissolving an appropriate amount of citric acid monohydrate (e.g., about 2.0 g/L) and adjusting the pH with a suitable base, e.g., NaOH, as described herein. Also in such embodiments, an additional chelating agent may include any suitable chelating agent such as, for example, a sugar alcohol, as described herein. For example, the additional chelating agent may be prepared dissolving, e.g., from about 30 g/L to about 3000 g/L xylitol or, e.g., about 300 g/L xylitol, in the water as described herein. Such embodiments may thus include preparing a chelating agent by dissolving about 2134 mg/L trisodium citrate dihydrate and about 475 mg/L citric acid monohydrate, or by producing an equivalent amount of citric acid and citrate in situ as described herein, and including an additional chelating agent by dissolving about 300 g/L xylitol in the water as described herein.

The method of the invention accordingly may include, for example, dissolving in a suitable solvent, e.g., water, about 2215 mg/L zinc sulfate heptahydrate, about 540 mg/L ferric chloride hexahydrate, about 19.8 mg/L manganese chloride tetrahydrate, about 102.3 mg/L copper chloride dihydrate, about 17.3 mg/L sodium selenite, about 4.9 mg/L sodium molybdate dihydrate, about 16.6 mg/L potassium iodide, about 5.3 mg/L chromic chloride hexahydrate, about 210 mg/L sodium fluoride, a chelating agent formed by dissolving about 2134 mg/L trisodium citrate dihydrate and about 475 mg/L citric acid monohydrate (or by forming in situ a chelating agent equivalent to about 2134 mg/L trisodium citrate dihydrate and about 475 mg/L citric acid monohydrate by dissolving the appropriate quantity of citric acid and adjusting the pH with NaOH as described herein), and about 300 g/L xylitol, in the water as described herein.

The method of the invention may include optionally adjusting the pH with a suitable acid (e.g., hydrochloric acid or sulfuric acid) and/or a suitable base (e.g., sodium hydroxide) as needed to achieve a pH of about 3.0-4.5, e.g., pH 3.0-4.4, pH 3.5-4.0 or pH 3.6-4.0, e.g., pH 3.6-4.0 with a target pH of about pH 3.8, as described herein. The method thus may include optionally adjusting the pH with an acid, e.g., hydrochloric acid or sulfuric acid, and/or a base, e.g., sodium hydroxide, as needed to achieve a pH of about 3.0-4.5, e.g., pH 3.0-4.4, pH 3.5-4.0 or pH 3.6-4.0, e.g., pH 3.6-4.0 with a target pH of about pH 3.8. In some embodiments, the method may include optionally adjusting the pH of the formulation as needed to about pH 3.6-4.0, or optionally adjusting the pH of the formulation as needed to achieve a target (e.g., in-process target) pH of about pH 3.8. In certain embodiments, the pH is adjusted with hydrochloric acid, and no sulfuric acid is used.

The method of the invention may include sterilizing the formulation by filtration, e.g., filtering the formulation through one or more sterilizing filters, and then filling the sterilized formulation into one or more sterile primary containers under aseptic conditions. Alternatively, or additionally, the method of the invention may include sterilizing the formulation by terminal sterilization, e.g., autoclaving, of the formulation packaged in one or more primary containers, e.g., vials. In certain embodiments, a packaged formulation according to the present invention is autoclaved at a temperature of 110° C.-122° C. for 5-50 minutes, e.g., for 10-40 minutes. In some embodiments, the thermal sterilization is carried out at a temperature of about 122° C. for 5-15 minutes. In other embodiments, the thermal sterilization is carried out at a temperature of about 115° C. for 30-40 minutes.

Any grade of water suitable for preparing injectable or parenteral formulations may be used as an aqueous solvent in the method of the invention. Suitable grades of water may include, for example, sterile, non-pyrogenic grades of water used in the preparation of injectable or parenteral formulations, e.g., WFI. In some embodiments, the water used in the method of the invention includes sterile WFI.

In some embodiments, the iodide concentration in the formulation prepared by the method of the invention decreases by no more than about 5% after autoclaving, e.g., at about 115° C. for about 30-40 minutes. For example, the iodide concentration in the formulation prepared by the method of the invention may decrease by no more than about 4% after autoclaving, or may decrease by no more than about 3% after autoclaving, or may decrease by no more than about 2% after autoclaving, or may decrease by no more than about 1% after autoclaving.

The invention also includes embodiments in which iodide concentration in the formulation prepared by the method of the invention decreases by no more than about 5% after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. For example, the iodide concentration in the formulation prepared by the method of the invention may decrease by no more than about 4%, or may decrease by no more than about 3%, or may decrease by no more than about 2%, or may decrease by no more than about 1%, after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

The method of the invention thus avoids the need to include any overage of iodide beyond the labeled content, and allows for end product to have a tighter product specification (e.g., 90-110% (±10%)) of the labeled content as compared to conventional Addaven™ which may require adding a 20% overage of iodide and/or a wider product specification of ±20% of the labeled/added content, e.g., to account for iodide loss.

In some embodiments, the fluoride concentration in the formulation prepared by the method of the invention decreases by no more than about 5% after autoclaving, e.g., at about 115° C. for about 30-40 minutes. For example, the fluoride concentration in the formulation prepared by the method of the invention may decrease by no more than about 4% after autoclaving, or may decrease by no more than about 3% after autoclaving, or may decrease by no more than about 2% after autoclaving, or may decrease by no more than about 1% after autoclaving.

The invention also includes embodiments in which the fluoride concentration in the formulation prepared by the method of the invention decreases by no more than about 5% after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. For example, the fluoride concentration in the formulation prepared by the method of the invention may decrease by no more than about 4%, or may decrease by no more than about 3%, or may decrease by no more than about 2%, or may decrease by no more than about 1% after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

In some embodiments, the formulation prepared by the method of the invention may contain, or may fall within a product specification requiring, 90%-110% of a labeled amount of each of the at least one trace elements present in the formulation after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

In some embodiments, the formulation prepared by the method of the invention is not more intensively colored than reference solution Greenish Yellow 1 (GY1) as determined by Ph.Eur.2.2.2 or USP<1061> after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

In some embodiments, the pH of the formulation prepared by the method of the invention changes by no more than about 0.5 pH units after autoclaving, e.g., at about 115° C. for about 30-40 minutes. For example, the pH of the formulation prepared by the method of the invention may change by no more than about 0.4 pH units after autoclaving, or may change by no more than about 0.3 pH units after autoclaving, or may change by no more than about 0.2 pH units after autoclaving, or may change by no more than about 0.1 pH units after autoclaving.

In some embodiments, the pH of the formulation prepared by the method of the invention changes by no more than about 0.5 pH units after storing the formulation at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. For example, the pH of the formulation prepared by the method of the invention may change by no more than about 0.4 pH units, or may change by no more than about 0.3 pH units, or may change by no more than about 0.2 pH units, or may change by no more than about 0.1 pH units, after storing the formulation at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

In some embodiments, the formulation prepared by the method of the invention is essentially non-corrosive to stainless steel. For example, the formulation prepared by the method of the invention may contain no more than about 0.1 mg/L of nickel after incubating the formulation in the presence of stainless steel for about 4 weeks at room temperature. The method of the invention may thus be performed with stainless steel manufacturing equipment, allowing for the production of the inventive formulation in stainless steel tanks with minimal risks to both product and patients due to corrosion.

The invention also includes embodiments in which the formulation prepared by the method of the invention is clear after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

In some embodiments, the formulation prepared by the method of the invention is essentially free from visible particles as determined by USP<790> and comprises, per container, not more than 6000 subvisible particles greater than or equal to 10 m and not more than 600 subvisible particles greater than or equal to 25 μm as determined by USP<788> after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. In such embodiments, the formulation prepared by the method of the invention may be essentially free from visible particles as determined by USP<790>, and may contain, per container, not more than 600, e.g., not more than 500, not more than 400, not more than 300, not more than 200, not more than 100, not more than 50, not more than 40, or not more than 30, subvisible particles greater than or equal to 10 μm, and not more than 60, e.g., not more than 50, not more than 40, not more than 30, not more than 20, or not more than 10, subvisible particles greater than or equal to 25 am as determined by USP<788>, after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months. In such embodiments, the formulation prepared by the method of the invention may remain clear after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

In some embodiments, the formulation prepared by the method of the invention contains not more than about 250 mcg/L aluminum, e.g., not more than about 200 mcg/L aluminum, not more than about 150 mcg/L aluminum, not more than about 100 mcg/L aluminum, not more than about 75 meg/L aluminum, or not more than about 50 meg/L aluminum, as determined, e.g., by a GFAAS or ICP-SFMS method, after storage at about 25° C. for about 3-24 months, at about 30° C. for about 3-12 months, and/or at about 40° C. for about 1-6 months without autoclaving, or after autoclaving, e.g., at about 115° C. for about 30-40 minutes, and then storing at about 25° C. for about 3-24 months, at about 30° C. for about 3-12 months, and/or at about 40° C. for about 1-6 months.

The invention additionally provides a pharmaceutical product which includes a container and the formulation of the invention (including the formulation prepared by the method of the invention) contained therein. The container may be sealed with any seal/closure suitable for intravenous or parenteral pharmaceutical solutions. The container may simply include a primary container, e.g., as a single container, or may include a primary container combined with one or more secondary containers, e.g., which may serve to protect, surround, and/or contain the primary container. The primary container and/or seal/closure may be made of any suitable material such as, for example, glass, plastic (e.g., polypropylene), and/or rubber. If desired, one or more surfaces of the primary container and/or seal/closure may be treated with a compound to limit reactivity of the primary container material with one or more components of the formulation. For example, the primary container and/or seal/closure may be treated with a silicon compound. The primary container also may be treated with ammonium sulfate. In some embodiments, the seal/closure may include bromobutyl or chlorobutyl rubber which may be optionally coated with a fluoropolymer. The primary container may be clear or opaque, or may be of any color, and/or may be colorless. For example, the primary container may be flint colored, or may be amber colored.

In some embodiments, the primary container may include a syringe. The syringe barrel may include or be made of, e.g., glass or plastic. A suitable plastic syringe may include a syringe barrel which includes or is made of an organic polymer such as, e.g., a polyethylene, a polypropylene, a cyclic olefin polymer (COP), or a cyclic olefin copolymer (COC). In some embodiments, the plastic syringe barrel may include COC. The invention also includes embodiments in which the plastic syringe barrel includes an amorphous COC that is copolymerized from norbomene and ethylene using a metallocene catalyst, such as, e.g., TOPAS™ COC manufactured by Topas Advanced Polymers GmbH. Non-limiting examples of syringes suitable for use in the present invention are described in US Patent Application Publication No. 2015/0273133A1, which is incorporated herein by reference in its entirety.

The primary container also may include, for example, a flexible, multi-layered bag. The bag may include a material which is chemically inert to the formulation, sterilizable, and weldable. Such materials include, without limitation, polyolefin polymers (e.g., a polyethylene or polypropylene), COP or COC, polycarbonates, styrene polymers, and block co-polymers thereof. In some embodiments, a polyolefin may be combined with an elastomeric polymer, such as, e.g., a styrene-ethylene/butylene-styrene-triblock polymer (SEBS), a styrene-ethylene/propylene-styrene-triblock polymer (SEPS), a styrene-butadiene-styrene-triblock polymer (SBS), and/or a styrene-isoprene-styrene triblock polymer (SIS). In certain embodiments, the innermost layer of the multi-layered bag comprises polypropylene and SEBS. In other embodiments, the innermost layer of the multi-layered bag comprises a polymer of cyclic olefin such as cycloolefin homopolymer or cycloolefin copolymer or mixture thereof. In yet other embodiments, the innermost layer of the multi-layered bag comprises ethylene-vinyl acetate copolymer. Suitable flexible bags are described in U.S. Pat. Nos. 5,783,269, 7,875,016, 8,162,915, 7,828,787, and/or 8,118,802, which are incorporated herein by reference in their entireties, and marketed under the tradename, FREEFLEX™. Other flexible polymeric containers suitable for use with a formulation according to the invention include, without limitation, GALAXY™, VIAFLO™, INTRAVIA™, and EXCEL™ containers.

In some embodiments, the primary container is disposed within and/or enclosed by a secondary container, such as a blister package or an overwrap. The secondary container may include an overwrap with, e.g., a first foil, a second foil, and a seal disposed along a common peripheral edge of the first and second foils. The first and second foils of the secondary container overwrap may include multilayer films. In some embodiments, the secondary container may be fully transparent to enable visual inspection of the primary container, labeling, and any other contents within the secondary container (e.g., oxygen absorber). The invention also includes embodiments in which the secondary container is fully intransparent, for example, an aluminum overpouch. The invention additionally includes embodiments in which the secondary container includes a completely or partially intransparent first foil and a completely or partially transparent second foil. Examples of secondary containers suitable for use in the present invention are described in US Patent Application Publication Nos. 2006/0240204 and 2019/0151202, which are incorporated herein by reference in their entireties.

The product of the invention may further include an oxygen absorber that absorbs and removes or decreases the level of oxygen that may be present in the formulation, in the headspace of the primary container, and/or within the secondary container after initial packaging, as well as oxygen that may permeate through the secondary container during the shelf life of the pharmaceutical product. The oxygen absorber may be provided in any suitable size, form, or shape including, for example, a sachet, pouch, capsule, label, strip, patch, canister, cartridge, lining, or sticker, etc. The oxygen absorber may be placed inside of the secondary container or adhered or integrated into the primary container and/or the secondary container. In some embodiments, the oxygen absorber may be in the form of a sachet or in the form of a canister. The pharmaceutical product of the invention also may include embodiments in which the oxygen absorber may be in the form of a label or in the form of a strip. The pharmaceutical product of the invention additionally may include embodiments in which the oxygen absorber may be in the form of a sticker or label that adheres to the secondary container or to the primary container. The pharmaceutical product of the invention further may include embodiments in which the oxygen absorber may be incorporated as part of the secondary container itself such as, for example, as part of a lid, film, or seal of the secondary container.

Suitable materials for oxygen absorbers may include, for example, metal-based substances that remove oxygen by reacting with it by chemical bonding, generally forming a metal oxide component. Metal-based substances may include, e.g., elemental iron as well as iron oxide, iron hydroxide, iron carbide, and the like, and combinations thereof. Other metals for use as oxygen absorbers may include, e.g., nickel, tin, copper, zinc, and combinations thereof. Metal-based oxygen absorbers may be provided in the form of a powder, e.g., to increase active surface area. Powder forms of suitable metal-based oxygen absorbers may be obtained by any known method including, but not limited to, atomization, milling, pulverization, and electrolysis. Additional materials for oxygen absorbers may include, e.g., low molecular weight organic compounds such as, e.g., ascorbic acid, sodium ascorbate, catechol and phenol, activated carbon, polymeric materials incorporating a resin and a catalyst, and combinations thereof. The oxygen absorber also may include a metal-based oxygen absorber, such as an iron-based oxygen absorber.

The pharmaceutical product of the invention may be packaged in a 10 mL dosage form, e.g., which includes about 10 mL of formulation, or the equivalent thereof, e.g., when combined with a diluent, contained within the container. In other embodiments, the pharmaceutical product of the invention may be packaged in a 100 mL dosage form, e.g., which includes about 100 mL of formulation, or the equivalent thereof, e.g., when combined with a diluent, contained within the container.

In some embodiments, the container of the product of the invention is a vial, ampoule, syringe, or bag. The product of the invention thus includes embodiments in which the container is a vial such as, for example, a plastic vial, e.g., a 10 mL polypropylene vial or a 100 mL polypropylene vial, or a glass vial, e.g., a 10 mL glass vial or a 100 mL glass vial.

Thus the invention provides a pharmaceutical product comprising a container and a formulation contained therein, wherein the formulation comprises chromium, copper, fluoride, iodine, iron, manganese, molybdenum, selenium, and zinc; a chelating agent, optionally an acid and/or a base as needed to adjust the pH, and water, wherein the formulation has a pH of about 3.0-4.5.

In some embodiments of the pharmaceutical product, the chelating agent comprises a polycarboxylic acid, a conjugate base, and a sugar alcohol, e.g., as described hereinabove. In certain embodiments, the formulation comprises about 1 mcg/mL chromium, about 38 mcg/mL copper, about 95 mcg/mL fluoride, about 13 mcg/mL iodine, about 0.11 mg/mL iron, about 5.5 mcg/mL manganese, about 1.9 mcg/mL molybdenum, about 7.9 mcg/mL selenium, and about 0.5 mg/mL zinc. In some embodiments, the zinc is provided as a sulfate salt.

In some embodiments of the pharmaceutical product, about 10 mL or about 100 mL of the formulation is contained within the container, and the container is a polypropylene vial.

The formulation in the pharmaceutical product of the invention preferably has the same stability characteristics as described hereinabove for a formulation of the invention. In certain embodiments of the pharmaceutical product, the formulation comprises 90%-110% of a labeled amount of each of the trace elements present in the formulation after storage of the product at room temperature, intermediate, and/or accelerated conditions for about 3-24 months. In some embodiments, the formulation does not contain any overage of iodide beyond a labeled amount.

In some embodiments, the formulation in the product of invention is clear after storage of the product at about 25° C. for about 3-24 months, at about 30° C. for about 3-12 months, and/or at about 40° C. for about 1-6 months.

In some embodiments, the formulation in the product of invention contains not more than about 250 mcg/L aluminum, e.g., not more than about 200 mcg/L aluminum, not more than about 150 mcg/L aluminum, not more than about 100 mcg/L aluminum, not more than about 75 mcg/L aluminum, or not more than about 50 mcg/L aluminum, as determined, e.g., by a GFAAS or ICP-SFMS method, after storage of the product at about 25° C. for about 3-24 months, at about 30° C. for about 3-12 months, and/or at about 40° C. for about 1-6 months.

In some embodiments, the formulation in the product of invention is essentially free from visible particles as determined by USP<790> and contains, per container, not more than 6000 subvisible particles greater than or equal to 10 μm and not more than 600 subvisible particles greater than or equal to 25 μm as determined by USP<788> after storage of the product at about 25° C. for about 3-24 months, at about 30° C. for about 3-12 months, and/or at about 40° C. for about 1-6 months. In such embodiments, the formulation in the product may be essentially free from visible particles as determined by USP<790>, and may contain, per container, not more than 600 (e.g., not more than 500, not more than 400, not more than 300, not more than 200, not more than 100, not more than 50, not more than 40, not more than 30) subvisible particles greater than or equal to 10 μm, and not more than 60 (e.g., not more than 50, not more than 40, not more than 30, not more than 20, or not more than 10) subvisible particles greater than or equal to 25 μm as determined by USP<788>, after storage at about 25° C. for about 3-24 months, at about 30° C. for about 3-12 months, and/or at about 40° C. for about 1-6 months. Also in such embodiments, the formulation in the product of the invention may be clear after storage of the product at about 25° C. for about 3-24 months, at about 30° C. for about 3-12 months, and/or at about 40° C. for about 1-6 months.

The invention further provides a method for treating or preventing trace element deficiency in a patient, which method includes administering to the patient a therapeutically effective amount of the formulation of the invention, including the formulation prepared by the method of the invention, and the formulation contained in the product of the invention, as described herein. The formulation of the invention may be administered by any suitable method, including any methods prescribed for the administration of conventional Addaven™. Suitable methods of administration may include, for example, intravenous injection or infusion. The formulation of the invention may be administered to treat any indication for which conventional Addaven™ may be approved. The formulation of the invention may be administered directly to the patient without prior dilution, although, preferably, the formulation may be diluted before administration to the patient.

The therapeutic method of the invention may thus include diluting the formulation with a suitable diluent before administration to the patient. The formulation may be diluted by a factor of, for example, from about 1:50 to about 1:100 in the diluent. Suitable diluents may include, for example, sterile water for injection, a sterile saline solution, a sterile solution containing glucose, or a sterile parenteral nutritional solution containing nutrients suitable for patients requiring parenteral nutrition. Such diluents may include, without limitation, any parenteral nutritional solution(s)/emulsion(s) that may be prescribed or otherwise suitable for co-administration with conventional Addaven™. In some embodiments, the diluent may include a parenteral nutritional solution/emulsion containing, for example, one or more proteins, electrolytes, and essential fatty acids, or a parenteral nutritional solution/emulsion containing, e.g., one or more proteins, fats, carbohydrates, salts/electrolytes, and vitamins.

The following examples further illustrate the invention but, of course, should not be construed as in any way limiting its scope.

### Example 1

This example demonstrates a method of preparing an exemplary product of the invention.

Into a suitable jacketed stainless-steel batch (mixing) tank equipped with a light-sensitive tank lid and a bottom-mounted mixer is added 100 L of Water for Injection. The batch tank cover should be closed at all times except when adding components and inspecting for dissolution.

426.8 g Sodium Citrate Dihydrate (trisodium citrate), USP, is added to the Water for Injection at a temperature of 30-35° C. while mixing, and the composition is mixed at a temperature of about 30-35° C. and a mixing speed of about 50-390 rpm until dissolution is complete (at least about 5 minutes).

95 g of Citric Acid Monohydrate, USP, is added at a temperature of about 30-35° C. while mixing, and the composition is mixed at a temperature of about 30-35° C. and a mixing speed of about 50-390 rpm until dissolution is complete (at least about 5 minutes).

Alternatively, the chelating agent solution is prepared by dissolving citric acid (e.g., Citric Acid Monohydrate USP) under similar conditions (at a temperature of about 30-35° C. and a mixing speed of about 50-390 rpm until dissolution is complete (at least about 5 minutes)), and adjusting the pH to 3.6-4.0 (target pH 3.8) with, e.g., 4% NaOH. The amount of citric acid added (e.g., 2.0 g/L citric acid monohydrate) is sufficient to achieve a chelating agent which is equivalent to about 426.8 g Sodium Citrate Dihydrate, USP, and 95 g of Citric Acid Monohydrate, USP, in the final volume of Water for Injection when the formulation including all components is adjusted to pH 3.6-4.0 (target pH 3.8).

60 kg of Xylitol is added to the citrate solution at a temperature of about 30-35° C. while mixing, and the composition is mixed at a temperature of about 30-35° C. and a mixing speed of about 50-390 rpm until dissolution is complete (at least about 5 minutes).

443 g of Zinc Sulfate Heptahydrate is added at a temperature carefully maintained at about 20-30° C. (target 25° C.) while mixing, and the composition is mixed at a temperature carefully maintained at about 20-30° C. (target 25° C.), and at a mixing speed of about 50-390 rpm, until dissolution is complete (at least about 5 minutes).

108 g of Iron (III) Chloride (Ferric Chloride) Hexahydrate is added while mixing, e.g., at a temperature of about 20-30° C., and the composition is mixed, e.g., at a temperature of about 20-30° C., and at a mixing speed of about 50-390 rpm, until dissolution is complete (at least about 5 minutes).

3.958 g of Manganese (II) Chloride Tetrahydrate, USP, is added at a temperature of about 20-30° C. while mixing, and the composition is mixed at a temperature of about 20-30° C. and a mixing speed of about 50-390 rpm until dissolution is complete (at least about 5 minutes).

20.46 g of Copper (II) Chloride Dihydrate, USP, is added at a temperature of about 20-30° C. while mixing, and the composition is mixed at a temperature of about 20-30° C. and a mixing speed of about 50-390 rpm until dissolution is complete (at least about 5 minutes).

3.458 g of Sodium Selenite is added at a temperature of about 20-30° C. while mixing, and the composition is mixed at a temperature of about 20-30° C. and a mixing speed of about 50-390 rpm until dissolution is complete (at least about 5 minutes).

0.970 g of Sodium Molybdate Dihydrate is added at a temperature of about 20-30° C. while mixing, and the composition is mixed at a temperature of about 20-30° C. and a mixing speed of about 50-390 rpm until dissolution is complete (at least about 5 minutes).

3.320 g of Potassium Iodide, USP, is added at a temperature of about 20-30° C. while mixing, and the composition is mixed at a temperature of about 20-30° C. and a mixing speed of about 50-390 rpm until dissolution is complete (at least about 5 minutes).

1.066 g of Chromic Chloride Hexahydrate, USP, is added at a temperature of about 20-30° C. while mixing, and the composition is mixed at a temperature of about 20-30° C. and a mixing speed of about 50-390 rpm until dissolution is complete (at least about 5 minutes).

42 g of Sodium Fluoride, USP, is added at a temperature of about 20-30° C. while mixing, and the composition is mixed at a temperature of about 20-30° C. and a mixing speed of about 50-390 rpm until dissolution is complete (at least about 5 minutes).

Water for Injection, in an amount sufficient to achieve a final volume of 200 L, is added while mixing at a temperature of about 20-30° C., and the composition is mixed at a temperature of about 20-30° C. and a mixing speed of about 50-390 rpm for about 15 minutes. The formulation has a final weight of about 219.88 kg.

If needed, the pH is adjusted with 10% HCl or 4% NaOH to achieve a pH of 3.6-4.0, with a target pH of 3.8.

The mixing is stopped, and the final formulation is pumped through a filter and into a suitable carboy while protecting the formulation from exposure to light during filtration.

The filtered formulation is filled into 10 cc plastic, e.g., polypropylene, vials (each containing 10 mL of formulation) or into 100 cc plastic vials (each containing 100 mL of formulation) while minimizing exposure to light, and each vial is sealed with a suitable stopper and capped.

The sealed, capped vials are autoclaved at 115° C. for about 30 minutes for 10 mL quantities, or at 115° C. for about 40 minutes for 100 mL quantities, to sterilize the product. The proposed specification (acceptable limits) for the product of the invention is 90-110% (±10%) of the labeled/added content for the active components.

### Example 2

This example demonstrates a method of preparing an exemplary product of the invention.

Into a suitable jacketed stainless-steel batch (mixing) tank equipped with a light-sensitive tank lid and a bottom-mounted mixer is added 200 L of Water for Injection. The batch tank cover should be closed at all times except when adding components and inspecting for dissolution.

853.6 g Sodium Citrate Dihydrate (trisodium citrate), USP, is added to the Water for Injection at a temperature of 30-35° C. while mixing, and the composition is mixed at a temperature of about 30-35° C. and a mixing speed of about 50-390 rpm until dissolution is complete (at least about 5 minutes).

190 g of Citric Acid Monohydrate, USP, is added at a temperature of about 30-35° C. while mixing, and the composition is mixed at a temperature of about 30-35° C. and a mixing speed of about 50-390 rpm until dissolution is complete (at least about 5 minutes).

Alternatively, the chelating agent solution is prepared by dissolving citric acid (e.g., Citric Acid Monohydrate USP) under similar conditions (at a temperature of about 30-35° C. and a mixing speed of about 50-390 rpm until dissolution is complete (at least about 5 minutes)), and adjusting the pH with, e.g., 4% NaOH. The amount of citric acid added (e.g., 2.0 g/L citric acid monohydrate) is sufficient to achieve a chelating agent which, when adjusted to pH 3.6-4.0 (target pH 3.8) after all components are added, is equivalent to about 853.6 g Sodium Citrate Dihydrate, USP, and 190 g of Citric Acid Monohydrate, USP, in the final volume of Water for Injection.

120 kg of Xylitol is added to the citrate solution at a temperature of about 30-35° C. while mixing, and the composition is mixed at a temperature of about 30-35° C. and a mixing speed of about 50-390 rpm until dissolution is complete (at least about 5 minutes).

886 g of Zinc Sulfate Heptahydrate is added at a temperature carefully maintained at about 20-30° C. (target 25° C.) while mixing, and the composition is mixed at a temperature carefully maintained at about 20-30° C. (target 25° C.), and at a mixing speed of about 50-390 rpm, until dissolution is complete (at least about 5 minutes).

216 g of Iron (III) Chloride (Ferric Chloride) Hexahydrate is added while mixing, e.g., at a temperature of about 20-30° C., and the composition is mixed, e.g., at a temperature of about 20-30° C., and at a mixing speed of about 50-390 rpm, until dissolution is complete (at least about 5 minutes).

7.916 g of Manganese (II) Chloride Tetrahydrate, USP, is added at a temperature of about 20-30° C. while mixing, and the composition is mixed at a temperature of about 20-30° C. and a mixing speed of about 50-390 rpm until dissolution is complete (at least about 5 minutes).

40.92 g of Copper (II) Chloride Dihydrate, USP, is added at a temperature of about 20-30° C. while mixing, and the composition is mixed at a temperature of about 20-30° C. and a mixing speed of about 50-390 rpm until dissolution is complete (at least about 5 minutes).

6.916 g of Sodium Selenite is added at a temperature of about 20-30° C. while mixing, and the composition is mixed at a temperature of about 20-30° C. and a mixing speed of about 50-390 rpm until dissolution is complete (at least about 5 minutes).

1.940 g of Sodium Molybdate Dihydrate is added at a temperature of about 20-30° C. while mixing, and the composition is mixed at a temperature of about 20-30° C. and a mixing speed of about 50-390 rpm until dissolution is complete (at least about 5 minutes).

6.640 g of Potassium Iodide, USP, is added at a temperature of about 20-30° C. while mixing, and the composition is mixed at a temperature of about 20-30° C. and a mixing speed of about 50-390 rpm until dissolution is complete (at least about 5 minutes).

2.132 g of Chromic Chloride Hexahydrate, USP, is added at a temperature of about 20-30° C. while mixing, and the composition is mixed at a temperature of about 20-30° C. and a mixing speed of about 50-390 rpm until dissolution is complete (at least about 5 minutes).

84 g of Sodium Fluoride, USP, is added at a temperature of about 20-30° C. while mixing, and the composition is mixed at a temperature of about 20-30° C. and a mixing speed of about 50-390 rpm until dissolution is complete (at least about 5 minutes).

Water for Injection, in an amount sufficient to achieve a final volume of 400 L, is added while mixing at a temperature of about 20-30° C., and the composition is mixed at a temperature of about 20-30° C. and a mixing speed of about 50-390 rpm for about 15 minutes. The formulation has a final weight of about 439.76 kg.

If needed, the pH is adjusted with 10% HCl or 4% NaOH to achieve a pH of 3.6-4.0, with a target pH of 3.8.

The mixing is stopped, and the final formulation is pumped through a filter and into a suitable carboy while protecting the formulation from exposure to light during filtration.

The filtered formulation is filled into 100 cc plastic, e.g., polypropylene, vials (each containing 100 mL of formulation) while minimizing exposure to light, and each vial is sealed with a suitable stopper and capped.

The sealed, capped vials are autoclaved at 115° C. for about 40 minutes, to sterilize the product. The proposed specification (acceptable limits) for the product of the invention is 90-110% (±100%) of the labeled/added content for the active components.

### Example 3

This example demonstrates the stability of exemplary formulations of the invention.

Base solutions containing the salts and concentrations described in Examples 1 and/or 2 were prepared, except that no citric acid or citrate was included, and the pH was varied to produce test formulations of pH 2.3, 2.8, 3.0, 3.5, and 4.0 by adjusting the pH as needed with an acid (10% HCl or 4M H₂SO₄) and/or base (4% NaOH). Exemplary formulations of the invention containing the salts, concentrations, and citric acid and citrate described in Examples 1 and/or 2 were prepared, except that the pH was varied to produce test formulations of pH 2.5, 3.0, 3.5, 4.0, and 4.5 by adjusting the pH as needed with an acid (10% HCl or 4M H₂SO₄) and/or base (4% NaOH). The base and invention formulations were filled into 10-mL polypropylene vials while minimizing exposure to light, and each vial was sealed with a bromobutyl rubber stopper and capped. The sealed, capped vials were autoclaved at 117.5° C. for about 31 minutes. The base and invention formulations were subjected to various storage conditions, and the trace element content was determined initially (To, before storage) and following storage. The trace element content also was determined in a subset of invention formulations before autoclaving.

Fluoride content was determined by ISE, and the contents of the remaining trace elements were determined by ICP-MS.

The ISE (potentiometry) conditions were as follows.
Measurement system: Metrohm Titrando 808
Reference electrode: Metrohm 60750100: LL-ISE Reference WOC
Fluoride ion-selective electrode: Metrohm 60502150: Ion Sel. EL F WOC SGJ

| Parameter | Value | |
|---|---|---|
| Sensor | Fluoride ISE | |
| Temperature measurement | Automatic | |
| Stirring speed | 2 | |
| Measured value drift | 0.5 | mV/min |
| Min. waiting time | 10 | sec. |
| Max. waiting time | 215 | sec. |
| Measuring interval | 2.0 | sec. |

The ICP-MS conditions for the assay of the trace elements Cr, Cu, Fe, Mn, Mo, Se and Zn were as follows.
Sample Prep: dilute samples 1+99 in 1% v/v HNO₃
Instrument: mass spectrometer equipped with a MicroMist nebulizer, a Scott-type spray chamber, and Cu/Ni cones. 12% Ar/CO₂ was used as optional gas to ensure the same carbon load for the calibration standards and the samples.
Detection:
   He Modus: Cr m/z 52 & 53; Mn m/z 55; Fe m/z 56; Cu m/z 65; Zn m/z 66; Mo m/z 95
   He Flow: 5 mL/min.
   H₂ Modus: Se m/z 78
   H₂ Flow: 7 mL/min.
Internal standard (both modi): Ge m/z 72 & 74+In m/z 115 were added online via a t-piece in front of the nebulizer to all samples.

Trace element content is expressed as the percentage (%) relative to the amount added during compounding. The proposed product specification for the formulation of the invention is 90-110% of a labeled concentration of each trace element.

The results for the base solutions are summarized below in Table 1.

TABLE 1
Base (without citrate)

| | | Cr | Mn | Fe | Cu | Zn | Se | Mo | F | I |
|---|---|---|---|---|---|---|---|---|---|---|
| Conditions | pH | (%) | (%) | (%) | (%) | (%) | (%) | (%) | (%) | (%) |
| T₀ | 2.3 | 100 | 100 | 98 | 104 | 102 | 108 | 101 | 98 | 92 |
| 25° C., 4 weeks | 2.3 | 98 | 98 | 96 | 102 | 101 | 107 | 99 | 97 | 90 |
| 40° C., 4 weeks | 2.3 | 99 | 100 | 98 | 103 | 103 | 108 | 100 | 96 | 85 |
| 60° C., 4 weeks | 2.3 | 98 | 98 | 97 | 100 | 101 | 107 | 100 | 92 | 72 |
| 25° C., 3 months | 2.3 | 95 | 103 | 94 | 97 | 98 | 103 | 100 | 94 | 90 |
| 40° C., 3 months | 2.3 | 94 | 103 | 94 | 97 | 98 | 103 | 100 | 92 | 75 |
| T₀ | 2.8 | 99 | 99 | 98 | 105 | 103 | 108 | 101 | 98 | 96 |
| 25° C., 4 weeks | 2.8 | 98 | 97 | 96 | 103 | 102 | 107 | 100 | 97 | 96 |
| 40° C., 4 weeks | 2.8 | 98 | 98 | 97 | 104 | 102 | 108 | 100 | 97 | 93 |
| 60° C., 4 weeks | 2.8 | 99 | 99 | 98 | 106 | 104 | 109 | 101 | 94 | 92 |
| 25° C., 3 months | 2.8 | 95 | 104 | 94 | 101 | 99 | 102 | 101 | 94 | 93 |
| 40° C., 3 months | 2.8 | 93 | 101 | 92 | 98 | 96 | 103 | 99 | 94 | 95 |
| T₀ | 3.0 | 100 | 100 | 99 | 107 | 104 | 109 | 102 | 98 | 96 |
| 25° C., 4 weeks | 3.0 | 98 | 98 | 98 | 105 | 103 | 108 | 99 | 97 | 95 |
| 40° C., 4 weeks | 3.0 | 97 | 97 | 97 | 105 | 102 | 106 | 101 | 98 | 95 |
| 60° C., 4 weeks | 3.0 | 99 | 99 | 98 | 106 | 104 | 108 | 101 | 95 | 95 |
| 25° C., 3 months | 3.0 | 95 | 104 | 94 | 102 | 99 | 102 | 102 | 95 | 94 |
| 40° C., 3 months | 3.0 | 95 | 104 | 95 | 102 | 100 | 103 | 101 | 95 | 97 |
| T₀ | 3.5 | 98 | 97 | 97 | 105 | 103 | 107 | 100 | 97 | 98 |
| 25° C., 4 weeks | 3.5 | 98 | 98 | 98 | 106 | 103 | 105 | 100 | 96 | 97 |
| 40° C., 4 weeks | 3.5 | 97 | 97 | 97 | 103 | 103 | 107 | 100 | 97 | 96 |
| 60° C., 4 weeks | 3.5 | 99 | 99 | 99 | 104 | 106 | 107 | 101 | 94 | 98 |
| 25° C., 3 months | 3.5 | 94 | 103 | 93 | 100 | 98 | 102 | 100 | 95 | 99 |
| 40° C., 3 months | 3.5 | 93 | 102 | 93 | 100 | 98 | 101 | 100 | 95 | 98 |
| T₀ | 4.0 | 97 | 97 | 97 | 105 | 103 | 107 | 100 | 96 | 97 |
| 25° C., 4 weeks | 4.0 | 97 | 97 | 97 | 105 | 103 | 106 | 99 | 95 | 97 |
| 40° C., 4 weeks | 4.0 | 97 | 98 | 98 | 107 | 105 | 105 | 100 | 97 | 95 |
| 60° C., 4 weeks | 4.0 | 95 | 97 | 95 | 107 | 104 | 103 | 97 | 94 | 98 |
| 25° C., 3 months | 4.0 | 93 | 103 | 93 | 101 | 98 | 99 | 99 | 94 | 98 |
| 40° C., 3 months | 4.0 | 95 | 105 | 94 | 102 | 100 | 100 | 104 | 95 | 99 |

The results for the exemplary formulations of the invention are summarized below in Table 2.

TABLE 2
Invention (with citrate)

| | | Cr | Mn | Fe | Cu | Zn | Se | Mo | F | I |
|---|---|---|---|---|---|---|---|---|---|---|
| Conditions | pH | (%) | (%) | (%) | (%) | (%) | (%) | (%) | (%) | (%) |
| T₀ Before | 2.5 | 94 | 100 | 96 | 102 | 100 | 100 | 96 | 95 | 99 |
| Autoclaving | | | | | | | | | | |
| T₀ After | 2.5 | 95 | 100 | 96 | 103 | 101 | 99 | 98 | 94 | 100 |
| Autoclaving | | | | | | | | | | |
| 80° C., 5 days | 2.5 | 95 | 100 | 97 | 103 | 101 | 101 | 98 | 89 | 99 |
| 60° C., 4 weeks | 2.5 | 99 | 101 | 98 | 101 | 99 | 101 | 96 | 90 | 105 |
| 40° C., 3 months | 2.5 | 100 | 103 | 102 | 103 | 98 | 99 | 94 | 93 | 107 |
| T₀ Before | 3.0 | 94 | 100 | 95 | 102 | 100 | 99 | 97 | 96 | 98 |
| Autoclaving | | | | | | | | | | |
| T₀ After | 3.0 | 93 | 99 | 95 | 100 | 99 | 100 | 97 | 94 | 99 |
| Autoclaving | | | | | | | | | | |
| 80° C., 5 days | 3.0 | 96 | 101 | 98 | 104 | 101 | 103 | 100 | 90 | 102 |
| 60° C., 4 weeks | 3.0 | 100 | 99 | 97 | 99 | 98 | 102 | 97 | 92 | 107 |
| 40° C., 3 months | 3.0 | 98 | 103 | 102 | 103 | 98 | 96 | 94 | 92 | 109 |
| T₀ Before | 3.5 | 93 | 99 | 95 | 102 | 99 | 100 | 98 | 96 | 100 |
| Autoclaving | | | | | | | | | | |
| T₀ After | 3.5 | 94 | 99 | 95 | 102 | 99 | 101 | 97 | 94 | 100 |
| Autoclaving | | | | | | | | | | |
| 80° C., 5 days | 3.5 | 95 | 100 | 97 | 104 | 101 | 103 | 99 | 91 | 101 |
| 60° C., 4 weeks | 3.5 | 100 | 99 | 97 | 99 | 97 | 103 | 96 | 92 | 107 |
| 40° C., 3 months | 3.5 | 97 | 102 | 101 | 103 | 97 | 96 | 94 | 94 | 109 |
| T₀ Before | 4.0 | 93 | 98 | 94 | 100 | 98 | 100 | 98 | 96 | 100 |
| Autoclaving | | | | | | | | | | |
| T₀ After | 4.0 | 93 | 98 | 94 | 101 | 98 | 101 | 98 | 95 | 101 |
| Autoclaving | | | | | | | | | | |
| 80° C., 5 days | 4.0 | 94 | 99 | 96 | 102 | 99 | 102 | 99 | 92 | 100 |
| 60° C., 4 weeks | 4.0 | 98 | 99 | 96 | 99 | 97 | 102 | 97 | 94 | 104 |
| 40° C., 3 months | 4.0 | 97 | 103 | 102 | 103 | 97 | 98 | 96 | 95 | 107 |
| T₀ Before | 4.5 | 93 | 98 | 94 | 101 | 97 | 100 | 98 | 97 | 100 |
| Autoclaving | | | | | | | | | | |
| T₀ After | 4.5 | 92 | 98 | 94 | 100 | 98 | 101 | 98 | 94 | 99 |
| Autoclaving | | | | | | | | | | |
| 80° C., 5 days | 4.5 | 93 | 98 | 95 | 101 | 99 | 102 | 99 | 94 | 97 |
| 60° C., 4 weeks | 4.5 | 98 | 98 | 95 | 98 | 96 | 102 | 96 | 97 | 99 |
| 40° C., 3 months | 4.5 | 96 | 103 | 101 | 102 | 97 | 95 | 95 | 97 | 100 |

These results show that iodide is more stable in the formulations of the invention containing citrate, even at lower pH values.

### Example 4

This example demonstrates iodide stability in exemplary formulations of the invention.

Base solutions (10 mL samples) containing the salts and concentrations described in Examples 1 and/or 2 were prepared, except that no citric acid or citrate was included, and pH was varied to produce test formulations of pH 2.3, 2.8, 3.0, 3.5, and 4.0 by adjusting the pH as needed with an acid (10% HCl or 4M H₂SO₄) and/or base (4% NaOH). Base solutions were stored for 3 months at 25° C., and for 3 months at 40° C., and iodide content was determined by HPLC after storage, and is expressed as the percentage (%) relative to the amount added during compounding.

Exemplary formulations of the invention (10 mL samples) containing the salts, concentrations, and citric acid and citrate described in Examples 1 and/or 2 were prepared, except that the pH was varied to produce test formulations of pH 2.5, 3.0, 3.5, 4.0, and 4.5 by adjusting the pH as needed with an acid (10% HCl or 4M H₂SO₄) and/or base (4% NaOH). Iodide content was determined by HPLC, and is expressed as the percentage (%) relative to the amount added during compounding.

The HPLC conditions were as follows.
Column: Waters XBridge C18 3.5 μm; 4.6×100 mm
Mobile Phase: 12.78 g disodium hydrogen phosphate, 3.20 g Cetyltrimethylammonium chloride 25% dissolved in 2800 mL water and 980 mL Acetonitrile (pH 6.7, adjusted with phosphoric acid 85%)
Column temperature: 45° C.
Flow rate: 2.0 mL/min.
Injection volume: 20 μL
Autosampler temperature: 20° C.
Detection: 226 nm (bandwidth: 4 nm) measuring wavelength
   360 nm (bandwidth: 80 nm) reference wavelength
Stop time: 70 minutes

The results for the base and formulations of the invention are summarized below in Tables 3 and 4, respectively.

TABLE 3
(Base)

| | | Iodide Content |
|---|---|---|
| Conditions | pH | (% by HPLC) |
| 25° C, 3 months | 2.3 | 86 |
| 25° C, 3 months | 2.8 | 98 |
| 25° C, 3 months | 3.0 | 100 |
| 25° C, 3 months | 3.5 | 103 |
| 25° C, 3 months | 4.0 | 103 |
| 40° C, 3 months | 2.3 | 76 |
| 40° C, 3 months | 2.8 | 93 |
| 40° C, 3 months | 3.0 | 97 |
| 40° C, 3 months | 3.5 | 103 |
| 40° C, 3 months | 4.0 | 102 |

TABLE 4
(Invention)

| | Iodide Content (% by HPLC) | | | | |
|---|---|---|---|---|---|
| | pH 2.5 | pH 3.0 | pH 3.5 | pH 4.0 | pH 4.5 |
| T₀ Before Autoclaving | 101 | 101 | 100 | 101 | 100 |
| T₀ After Autoclaving | 101 | 102 | 100 | 100 | 100 |
| 80° C, 5 days | 101 | 101 | 101 | 101 | 97 |
| 60° C, 4 weeks | 102 | 102 | 103 | 99 | 91 |
| 40° C, 3 months | 99 | 101 | 100 | 97 | 89 |

These results show that the formulation of the invention provides greater iodide stability relative to base over a broad pH range.

### Example 5

This example demonstrates the stability of exemplary formulations of the invention with respect to particulates.

Base solutions containing the salts and concentrations described in Examples 1 and/or 2 were prepared, except that no citric acid or citrate was included, and pH was varied to produce test formulations of pH 3.0, 3.2, 3.4, 3.6, 3.8, and 4.0 by adjusting the pH as needed with an acid (10% HCl or 4M H₂SO₄) and/or base (4% NaOH).

Exemplary formulations of the invention containing the salts, concentrations, and citric acid and citrate described in Examples 1 and/or 2 were prepared, except that the pH was varied to produce test formulation of pH 2.5, 3.0, 3.5, 4.0, and 4.5 by adjusting the pH as needed with an acid (10% HCl or 4M H₂SO₄) and/or base (4% NaOH).

Particle counts for subvisible particles of >10 μm and >25 μm were determined by USP<788>, and visible particles were determined by USP<790> and assessed for compliance (where compliance means essentially free of visible particles), before and after storage under various conditions.

The results for the base solutions are summarized below in Tables 5 and 6.

TABLE 5
(Base)

| | Subvisible Particles ≥10 μm/Container | | | | | |
|---|---|---|---|---|---|---|
| | (≤6000/Container) | | | | | |
| | pH 3.0 | pH 3.2 | pH 3.4 | pH 3.6 | pH 3.8 | pH 4.0 |
| T₀ Before Storage | 34 | 46 | 28 | 10 | 50 | 382 |
| 50° C., 4 weeks | 28 | 50 | 220 | 204 | 412 | 858 |
| 60° C., 4 weeks | 44 | 152 | 260 | 614 | 1202 | 930 |
| 25° C., 5 months | 22 | 4 | 5 | 11 | 13 | 48 |

TABLE 6
(Base)

| | Subvisible Particles ≥25 μm/Container | | | | | |
|---|---|---|---|---|---|---|
| | (≤600/Container) | | | | | |
| | pH 3.0 | pH 3.2 | pH 3.4 | pH 3.6 | pH 3.8 | pH 4.0 |
| T₀ Before Storage | 2 | 0 | 4 | 2 | 2 | 14 |
| 50° C., 4 weeks | 0 | 2 | 28 | 14 | 20 | 32 |
| 60° C., 4 weeks | 0 | 2 | 2 | 22 | 88 | 16 |
| 25° C., 5 months | 4 | 0 | 0 | 0 | 1 | 2 |

The results for the formulations of the invention are summarized below in Tables 7 and 8.

TABLE 7
(Invention)

| | Subvisible Particles ≥10 μm/Container | | | | |
|---|---|---|---|---|---|
| | (≤6000/Container) | | | | |
| | pH 2.5 | pH 3.0 | pH 3.5 | pH 4.0 | pH 4.5 |
| T₀ Before Storage | 18 | 16 | 4 | 8 | 20 |
| 80° C, 5 days | 8 | 8 | 34 | 12 | 6 |
| 60° C, 4 weeks | 22 | 26 | 18 | 10 | 16 |
| 40° C, 3 months | 204 | 208 | 106 | 56 | 84 |

TABLE 8
(Invention)

| | Subvisible Particles ≥25 μm/Container | | | | |
|---|---|---|---|---|---|
| | (≤600/Container) | | | | |
| | pH 2.5 | pH 3.0 | pH 3.5 | pH 4.0 | pH 4.5 |
| T₀ Before Storage | 6 | 2 | 0 | 2 | 2 |
| 80° C, 5 days | 0 | 0 | 2 | 0 | 0 |
| 60° C, 4 weeks | 2 | 0 | 2 | 0 | 2 |
| 40° C, 3 months | 14 | 24 | 4 | 6 | 54 |

All base and invention formulations were essentially free of visible particles over the evaluated storage conditions, which were T₀; 50° C., 4 weeks; 60° C., 4 weeks; and 25° C., 5 months for exemplary base solutions and T₀; 80° C., 5 days; 60° C., 4 weeks; and 40° C., 3 months for exemplary invention formulations.

These results show that the formulation of the invention is more stable than base solutions with respect to subvisible particle formation. The base solutions exhibited an increase in subvisible particles after storage, particularly with increasing pH values. The formulations of the invention exhibited little to no increase in subvisible particles relative to the base solutions after storage across the entire pH range.

### Example 6

This example demonstrates the non-corrosiveness of exemplary formulations of the invention to stainless steel. Samples of the formulation of the invention containing the salts, concentrations, and chelating agent as described in Examples 1 and/or 2 (zinc sulfate heptahydrate as zinc source, citric acid and citrate, xylitol, pH 3.8), but which were not filtered or autoclaved, were compared with samples of conventional Addaven™.

To test for corrosiveness, samples of the formulation of the invention and conventional Addaven™, each containing about 20 mL of solution and each containing a manually welded stainless steel half pipe section, were incubated in 50 mL glass vials at room temperature for 4 weeks in a dark place. As a blank/control for each formulation, samples of the formulation of the invention and conventional Addaven™ (about 20 mL each) were filled into 50 mL glass vials without stainless steel, and were stored together with the incubation samples containing stainless steel.

Corrosiveness was assessed by observing color changes and measuring nickel content in the formulations relative to blanks (controls without stainless steel). Color changes and nickel content reflect the extent of corrosion. During incubation, the samples were regularly checked visually. The results for color changes and nickel content are summarized in Tables 9 and 10 respectively.

TABLE 9

| | Color | | | |
|---|---|---|---|---|
| | Conventional Addaven ™ | | Invention | |
| | (Zinc Chloride, No Citrate) | | (Zinc Sulfate, Citrate) | |
| | Blank/Control | Addaven ™ + | Blank/Control | Invention + |
| | (Addaven ™ Without | Stainless | (Invention Without | Stainless |
| Conditions | Stainless Steel) | Steel | Stainless Steel) | Steel |
| T₀ (Before | Colorless | Colorless | Yellow | Yellow |
| Incubation) | | | | |
| After 18 Days | Colorless | Slight | Yellow | Yellow |
| Incubation | | Yellow | | |
| After 4 Weeks | Colorless | Orange | Yellow | Yellow |
| Incubation | | Brownish | | |

TABLE 10

| | Nickel Content (mg/L) | | | |
|---|---|---|---|---|
| | Conventional Addaven ™ | | Invention | |
| | (Zinc Chloride, No Citrate) | | (Zinc Sulfate, Citrate) | |
| | Blank/Control | Addaven ™ + | Blank/Control | Invention + |
| | (Addaven ™ Without | Stainless | (Invention Without | Stainless |
| Conditions | Stainless Steel) | Steel | Stainless Steel) | Steel |
| After 4 Weeks | <0.005 | 31.5 | <0.005 | 0.086 |
| Incubation | | | | |

All control samples (without stainless steel) did not change color during storage and had nickel contents of <0.005 mg/L after 4 weeks of the incubation conditions. In the presence of stainless steel, the conventional Addaven™ formulation exhibited considerable discoloration, which intensified from slight yellow to orange-brownish, and had a nickel content of 31.5 mg/L, after 4 weeks of incubation with stainless steel. The formulation of the invention had a slight yellow color with or without stainless steel throughout the duration of the study and had a nickel content of only 0.086 mg/mL after 4 weeks of incubation with stainless steel.

These results show that the formulation of the invention is essentially non-corrosive to stainless steel. The formulation of the invention is thus compatible with stainless steel manufacturing equipment.

### Example 7

This example demonstrates the stability of the formulation of the invention with respect to pH changes. Exemplary formulations of the invention containing the salts, concentrations, and citric acid and citrate described in Examples 1 and/or 2 were prepared, except that the pH was varied to produce test formulations of pH 2.5, 3.0, 3.5, 4.0, and 4.5 by adjusting the pH as needed with an acid (10% HCl or 4M H₂SO₄) and/or base (4% NaOH). The pH was measured after autoclaving and after storage under various conditions. The results are summarized below in Table 11.

TABLE 11

| | pH 2.5 | pH 3.0 | pH 3.5 | pH 4.0 | pH 4.5 |
|---|---|---|---|---|---|
| T₀ After Autoclaving | 2.7 | 3.1 | 3.6 | 4.0 | 4.6 |
| 80° C, 5 days | 2.7 | 3.1 | 3.6 | 4.1 | 5.1 |
| 60° C, 4 weeks | 2.6 | 3.1 | 3.6 | 4.1 | 5.2 |
| 40° C, 3 months | 2.6 | 3.1 | 3.6 | 4.1 | 4.6 |

These results show that the formulation of the invention is resistant to substantial changes in pH due to autoclaving and under accelerated storage conditions, particularly over an initial pH range of 3.0-4.0.

### Example 8

This example demonstrates the stability of the formulation of the invention with respect to color changes.

Base solutions containing the salts and concentrations described in Examples 1 and/or 2 were prepared, except that no citric acid or citrate was included, and pH was varied to produce test formulations of different pH values by adjusting the pH as needed with an acid (10% HCl or 4M H₂SO₄) and/or base (4% NaOH).

Exemplary formulations of the invention containing the salts, concentrations, and citric acid and citrate described in Examples 1 and/or 2 were prepared, except that the pH was varied to produce test formulations of different pH values by adjusting the pH as needed with an acid (10% HCl or 4M H₂SO₄) and/or base (4% NaOH).

The degree of coloration was measured in autoclaved formulations before and after storage by USP<1061>.

The results for the base solutions (degree of coloration) are summarized below in Table 12.

TABLE 12
(Base)

| | Degree of Coloration | | | | |
|---|---|---|---|---|---|
| | pH 2.3 | pH 2.8 | pH 3.0 | pH 3.5 | pH 4.0 |
| T₀ | Y 6.1 | Y 5.2 | Y4.2 | Y 2.9 | Y <1.0 |
| | GY 6.3 | GY 4.5 | | | |
| | BY 6.2 | BY 4.9 | | | |
| 60° C, 4 weeks | Y 6.3 | Y4.5 | Y 4.8 | Y 1.8 | Y <1.0 |
| | GY 6.4 | GY 4.0 | BY 4.8 | | |
| | BY 6.3 | BY 4.5 | | | |
| 25° C, 3 months | Y 6.4 | Y 5.3 | Y 4.6 | Y 3.1 | Y <1.0 |
| | GY 6.5 | GY 5.2 | | | |
| | BY 6.4 | BY 5.4 | | | |
| 40° C, 3 months | Y 6.2 | Y 5.1 | Y4.2 | Y 2.6 | Y <1.0 |
| | GY 6.4 | GY 4.8 | | | |
| | BY 6.2 | BY 5.0 | | | |

The results for the exemplary formulations of the invention (degree of coloration) are summarized below in Table 13.

TABLE 13
(Invention)

| | Degree of Coloration | | | | |
|---|---|---|---|---|---|
| | pH 2.5 | pH 3.0 | pH 3.5 | pH 4.0 | pH 4.5 |
| T₀ | GY 3.0 | GY 2.6 | GY 2.3 | GY 2.0 | GY 2.0 |
| 80° C, 5 days | GY 2.2 | GY 2.4 | GY 2.2 | GY 2.0 | GY 1.9 |
| 60° C, 4 weeks | GY 2.0 | GY 2.0 | GY 2.0 | GY 1.9 | GY 1.9 |
| 40° C, 3 months | GY 0.9 | GY 1.8 | GY 3.1 | GY 2.0 | GY 2.4 |

The base solutions as shown in Table 12 exhibited changes in coloration at different pH values, even with relatively minor variations in pH. The formulations of the invention as shown in Table 13 exhibited no appreciable change in coloration across the entire pH range, either before or after storage under accelerated conditions. These results show that the formulation of the invention is remarkably stable to quantifiable changes in coloration over a wide pH range.

### Example 9

This example demonstrates the stability of the formulation of the invention as assessed using ICP-OES to quantify trace element concentrations.

Samples of the formulation of the invention containing the salts, concentrations, and chelating agent as described in Examples 1 and/or 2 were prepared, filled into plastic vials, stoppered, capped, autoclaved, and then and subjected to various storage conditions. The fluoride content was determined by ISE as described in Example 3, iodide content was determined by HPLC as described in Example 4, and contents of the remaining trace elements were determined by ICP-OES under the following conditions.

### Detection Wavelengths:

Ar (420.067 nm)
Cr (267.716 nm)
Cu (213.598 nm)
Fe (238.204 nm)
Mn (257.610 nm)
Mo (202.032 nm)
Sc (361.383 nm)
Se (196.026 nm)
Zn (328.233 nm)

### Common Conditions:

Replicates: 5
Pump speed: 12 RPM
Uptake delay: 30 sec.
Rinse time: 30 sec.

### Measurement Conditions:

Read time: 20 sec.
RF power: 1.20 kW
Stabilization time: 20 sec.
Viewing mode: radial
Viewing height: 8 nm
Nebulizer flow: 0.70 L/min
Plasma Flow: 12.0 L/min
Aux flow: 1.00 L/min
Make up flow: 0.00 L/min

The results for the formulations of the invention are summarized below in Table 14.

TABLE 14

| | Cr | Mn | Fe | Cu | Zn | Se | Mo | F | I |
|---|---|---|---|---|---|---|---|---|---|
| Conditions | (%) | (%) | (%) | (%) | (%) | (%) | (%) | (%) | (%) |
| 25° C., 3 months | 98 | 99 | 99 | 99 | 100 | 99 | 99 | 99 | 98 |
| 25° C., 6 months | 100 | 100 | 98 | 100 | 101 | 99 | 100 | 98 | 100* |
| 40° C., 3 months | 98 | 99 | 99 | 99 | 100 | 99 | 99 | 97 | 97 |
| 40° C., 6 months | 100 | 101 | 99 | 100 | 101 | 100 | 101 | 97 | 98* |

*storage for approximately 7.5 months

These results show that exemplary formulations of the invention are stable with respect to all trace elements over at least 3-6 months at 25° C. and 40° C. as determined by ICP-OES, ISE (F), and HPLC (I).

### Example 10

This example demonstrates the long-term stability of exemplary formulations of the invention packaged in 10-mL vials.

Samples of 10-mL plastic vials from three batches (Batches 1-3) prepared according to the methods described in Example 1 were subjected to room temperature, intermediate, and accelerated stability testing over a six- to nine-month period. The results of stability testing are set forth below in Tables 15 (room temperature), 16 (accelerated), and 17 (room temperature and intermediate, 9 months).

TABLE 15
Room Temperature Stability (25° C. ± 2° C./40% RH ± 5% RH), Upright

| | Acceptance | | Initial | | | 3 Months | | | 6 Months | | |
|---|---|---|---|---|---|---|---|---|---|---|---|
| Test | criteria | Units | Batch1 | Batch2 | Batch3 | Batch1 | Batch2 | Batch3 | Batch1 | Batch2 | Batch3 |
| Clarity | Clear | — | Clear | Clear | Clear | Clear | Clear | Clear | Clear | Clear | Clear |
| Coloration | ≤GY1* | — | GY2.0 | GY1.9 | GY1.8 | GY2.0 | GY2.1 | GY2.1 | GY1.9 | GY2.0 | GY1.9 |
| pH | 3.0-4.4 | — | 3.9 | 3.9 | 3.9 | 3.9 | 3.8 | 3.7 | 3.9 | 3.9 | 3.9 |
| Osmolality | 2520-3080 | mOsm/kg | 2751 | 2746 | 2783 | 2801 | 2746 | 2795 | 2732 | 2769 | 2770 |
| Particulates | ≤6000/vial | — | 17 | 28 | 27 | 22 | 13 | 15 | 1 | 1 | 0 |
| (≥10 μm) | | | | | | | | | | | |
| Particulates | ≤600/vial | — | 8 | 15 | 15 | 4 | 3 | 3 | 0 | 0 | 0 |
| (≥25 μm) | | | | | | | | | | | |
| Aluminum | ≤250 | μg/L | <20 | <20 | <20 | nt | nt | nt | <20** | <20** | <20** |
| Assay | | | | | | | | | | | |
| Iron | 1.8-2.2 | μmol/mL | 1.9 | 1.9 | 1.9 | 1.9 | 2.0 | 2.0 | 1.9 | 2.0 | 2.0 |
| Zinc | 6.9-8.5 | μmol/mL | 7.4 | 7.4 | 7.4 | 7.4 | 7.5 | 7.5 | 7.4 | 7.5 | 7.5 |
| Manganese | 0.09-0.11 | μmol/mL | 0.10 | 0.10 | 0.10 | 0.10 | 0.10 | 0.10 | 0.10 | 0.10 | 0.10 |
| Copper | 0.54-0.66 | μmol/mL | 0.59 | 0.59 | 0.59 | 0.59 | 0.59 | 0.59 | 0.59 | 0.59 | 0.59 |
| Selenium | 0.09-0.11 | μmol/mL | 0.10 | 0.10 | 0.10 | 0.10 | 0.10 | 0.10 | 0.10 | 0.10 | 0.10 |
| Molybdenum | 0.018-0.022 | μmol/mL | 0.019 | 0.019 | 0.020 | 0.020 | 0.020 | 0.020 | 0.020 | 0.020 | 0.020 |
| Chromium | 0.018-0.022 | μmol/mL | 0.019 | 0.019 | 0.019 | 0.019 | 0.019 | 0.020 | 0.019 | 0.019 | 0.020 |
| Iodide | 0.09-0.11 | μmol/mL | 0.10 | 0.10 | 0.10 | 0.10 | 0.10 | 0.10 | 0.10 | 0.10 | 0.10 |
| Fluoride | 4.5-5.5 | μmol/mL | 5.0 | 4.9 | 5.0 | 4.9 | 4.9 | 4.9 | 4.8 | 4.8 | 4.9 |

*Not more intensively colored than reference solution GY1;
nt, not tested;
**inverted sample

TABLE 16
Accelerated Stability (40° C. ± 2° C./NMT 25% RH), Upright

| | Acceptance | | 1 Month | | | 3 Months | | | 6 Months | | |
|---|---|---|---|---|---|---|---|---|---|---|---|
| Test | criteria | Units | Batch1 | Batch2 | Batch3 | Batch1 | Batch2 | Batch3 | Batch1 | Batch2 | Batch3 |
| Clarity | Clear | — | Clear | Clear | Clear | Clear | Clear | Clear | Clear | Clear | Clear |
| Coloration | ≤GY1* | — | GY3.3 | GY2.7 | GY2.7 | GY2.0 | GY2.2 | GY1.9 | GY1.9 | GY2.0 | GY1.8 |
| pH | 3.0-4.4 | — | 4.0 | 3.9 | 3.9 | 3.8 | 3.8 | 3.8 | 3.9 | 3.9 | 3.9 |
| Osmolality | 2520-3080 | mOsm/kg | 2723 | 2741 | 2769 | 2837 | 2782 | 2760 | 2797 | 2811 | 2816 |
| Particulates | ≤6000/vial | — | 2 | 6 | 3 | 1 | 5 | 1 | 1 | 3 | 2 |
| (≥10 μm) | | | | | | | | | | | |
| Particulates | ≤600/vial | — | 0 | 1 | 0 | 0 | 0 | 0 | 0 | 3 | 0 |
| (≥25 μm) | | | | | | | | | | | |
| Aluminum | ≤250 | μg/L | nt | nt | nt | nt | nt | nt | <20** | <20** | <20** |
| Assay | | | | | | | | | | | |
| Iron | 1.8-2.2 | μmol/mL | 1.9 | 1.9 | 1.9 | 2.0 | 2.0 | 2.0 | 2.0 | 2.0 | 2.0 |
| Zinc | 6.9-8.5 | μmol/mL | 7.4 | 7.5 | 7.4 | 7.4 | 7.5 | 7.6 | 7.5 | 7.6 | 7.6 |
| Manganese | 0.09-0.11 | μmol/mL | 0.10 | 0.10 | 0.10 | 0.10 | 0.10 | 0.10 | 0.10 | 0.10 | 0.10 |
| Copper | 0.54-0.66 | μmol/mL | 0.59 | 0.59 | 0.59 | 0.60 | 0.60 | 0.60 | 0.60 | 0.60 | 0.61 |
| Selenium | 0.09-0.11 | μmol/mL | 0.10 | 0.10 | 0.10 | 0.10 | 0.10 | 0.10 | 0.10 | 0.10 | 0.10 |
| Molybdenum | 0.018-0.022 | μmol/mL | 0.019 | 0.019 | 0.020 | 0.020 | 0.020 | 0.020 | 0.019 | 0.020 | 0.020 |
| Chromium | 0.018-0.022 | μmol/mL | 0.019 | 0.019 | 0.019 | 0.019 | 0.020 | 0.020 | 0.020 | 0.020 | 0.020 |
| Iodide | 0.09-0.11 | μmol/mL | 0.10 | 0.10 | 0.10 | 0.10 | 0.10 | 0.10 | 0.10 | 0.10 | 0.10 |
| Fluoride | 4.5-5.5 | μmol/mL | 4.9 | 4.9 | 4.9 | 4.9 | 4.9 | 4.9 | 4.8 | 4.7 | 4.8 |

*Not more intensively colored than reference solution GY1;
nt, not tested;
**inverted sample

TABLE 17
9 Months Room (25° C. ± 2° C./40% RH ± 5% RH) or Intermediate
(30° C. ± 2° C./65% RH ± 5% RH) Temperature, Upright

| | Acceptance | | Room Temperature | | | Intermediate | | |
|---|---|---|---|---|---|---|---|---|
| Test | criteria | Units | Batch1 | Batch2 | Batch3 | Batch1 | Batch2 | Batch3 |
| Clarity | Clear | — | Clear | Clear | Clear | Clear | Clear | Clear |
| Coloration | <GY1* | — | GY1.8 | GY1.8 | GY1.8 | GY1.8 | GY1.8 | GY1.8 |
| pH | 3.0-4.4 | — | 3.9 | 3.9 | 3.9 | 3.9 | 3.9 | 3.9 |
| Osmolality | 2520-3080 | mOsm/kg | 2756 | 2761 | 2787 | 2756 | 2765 | 2777 |
| Particulates | ≤6000/vial | — | 8 | 10 | 2 | 1 | 1 | 1 |
| (≥10 μm) | | | | | | | | |
| Particulates | ≤600/vial | — | 3 | 2 | 1 | 0 | 0 | 0 |
| (>25 μm) | | | | | | | | |
| Aluminum | ≤250 | μg/L | nt | nt | nt | nt | nt | nt |
| Assay | | | | | | | | |
| Iron | 1.8-2.2 | μmol/mL | 2.0 | 1.9 | 1.9 | 1.9 | 1.9 | 2.0 |
| Zinc | 6.9-8.5 | μmol/mL | 7.5 | 7.5 | 7.5 | 7.5 | 7.5 | 7.6 |
| Manganese | 0.09-0.11 | μmol/mL | 0.10 | 0.10 | 0.10 | 0.10 | 0.10 | 0.10 |
| Copper | 0.54-0.66 | μmol/mL | 0.59 | 0.58 | 0.59 | 0.59 | 0.59 | 0.59 |
| Selenium | 0.09-0.11 | μmol/mL | 0.10 | 0.10 | 0.10 | 0.10 | 0.10 | 0.10 |
| Molybdenum | 0.018-0.022 | μmol/mL | 0.020 | 0.019 | 0.020 | 0.020 | 0.020 | 0.020 |
| Chromium | 0.018-0.022 | μmol/mL | 0.019 | 0.019 | 0.019 | 0.019 | 0.019 | 0.020 |
| Iodide | 0.09-0.11 | μmol/mL | 0.10 | 0.10 | 0.10 | 0.10 | 0.10 | 0.10 |
| Fluoride | 4.5-5.5 | μmol/mL | 4.8 | 4.9 | 4.8 | 4.7 | 4.7 | 4.7 |

*Not more intensively colored than reference solution GY1;
nt, not tested

These results show that a trace element formulation according to the invention packaged in a 10-mL vial is stable under room temperature, intermediate, and accelerated storage conditions for at least six months or at least nine months.

### Example 11

This example demonstrates the long-term stability of exemplary formulations of the invention packaged in 100-mL vials.

Samples of 100-mL plastic vials from three batches (Batches 4-6) prepared according to the methods described in Example 1 (Batch 5) or Example 2 (Batches 4 and 6) were subjected to room temperature and accelerated stability testing over a six- to nine-month period. The results of stability testing are set forth below in Tables 18 (room temperature), 19 (accelerated), and 20 (room temperature and intermediate, 9 months).

TABLE 18
Room Temperature Stability (25° C. ± 2° C./40% RH ± 5% RH), Upright

| | Acceptance | | Initial | | | 3 Months | | | 6 Months | | |
|---|---|---|---|---|---|---|---|---|---|---|---|
| Test | criteria | Units | Batch4 | Batch5 | Batch6 | Batch4 | Batch5 | Batch6 | Batch4 | Batch5 | Batch6 |
| Clarity | Clear | — | Clear | Clear | Clear | Clear | Clear | Clear | Clear | Clear | Clear |
| Coloration | ≤GY1* | — | GY1.8 | GY1.8 | GY1.9 | GY1.8 | GY1.8 | GY1.8 | GY1.9 | GY1.9 | GY1.9 |
| pH | 3.0-4.4 | — | 3.9 | 3.9 | 3.9 | 3.9 | 3.9 | 3.9 | 3.9 | 3.9 | 3.9 |
| Osmolality | 2520-3080 | mOsm/kg | 2769 | 2793 | 2767 | 2746 | 2730 | 2791 | 2751 | 2740 | 2799 |
| Particulates | ≤6000/vial | — | 31 | 18 | 7 | 47 | 40 | 0 | 13 | 7 | 253 |
| (≥10 μm) | | | | | | | | | | | |
| Particulates | ≤600/vial | — | 16 | 5 | 3 | 20 | 27 | 0 | 7 | 0 | 100 |
| (≥25 μm) | | | | | | | | | | | |
| Aluminum | ≤250 | μg/L | <20 | <20 | <20 | nt | nt | nt | <20** | <20** | <20** |
| Assay | | | | | | | | | | | |
| Iron | 1.8-2.2 | μmol/mL | 2.0 | 2.0 | 2.0 | 2.0 | 1.9 | 1.9 | 1.9 | 2.0 | 2.0 |
| Zinc | 6.9-8.5 | μmol/mL | 7.5 | 7.5 | 7.5 | 7.5 | 7.4 | 7.4 | 7.4 | 7.4 | 7.4 |
| Manganese | 0.09-0.11 | μmol/mL | 0.10 | 0.10 | 0.10 | 0.10 | 0.10 | 0.10 | 0.10 | 0.10 | 0.10 |
| Copper | 0.54-0.66 | μmol/mL | 0.59 | 0.59 | 0.60 | 0.60 | 0.58 | 0.59 | 0.59 | 0.59 | 0.59 |
| Selenium | 0.09-0.11 | μmol/mL | 0.10 | 0.10 | 0.10 | 0.10 | 0.10 | 0.10 | 0.10 | 0.10 | 0.10 |
| Molybdenum | 0.018-0.022 | μmol/mL | 0.020 | 0.020 | 0.020 | 0.020 | 0.020 | 0.020 | 0.020 | 0.020 | 0.020 |
| Chromium | 0.018-0.022 | μmol/mL | 0.019 | 0.019 | 0.019 | 0.020 | 0.019 | 0.019 | 0.019 | 0.019 | 0.019 |
| Iodide | 0.09-0.11 | μmol/mL | 0.10 | 0.10 | 0.10 | 0.10 | 0.10 | 0.10 | 0.10 | 0.10 | 0.10 |
| Fluoride | 4.5-5.5 | μmol/mL | 5.0 | 5.0 | 5.0 | 4.9 | 5.0 | 5.0 | 5.0 | 4.9 | 4.9 |

*Not more intensively colored than reference solution GY1;
nt, not tested;
**inverted sample

TABLE 19
Accelerated Stability (40° C. ± 2° C./NMT 25% RH), Upright

| | Acceptance | | 1 Month | | | 3 Months | | | 6 Months | | |
|---|---|---|---|---|---|---|---|---|---|---|---|
| Test | criteria | Units | Batch4 | Batch5 | Batch6 | Batch4 | Batch5 | Batch6 | Batch4 | Batch5 | Batch6 |
| Clarity | Clear | — | Clear | Clear | Clear | Clear | Clear | Clear | Clear | Clear | Clear |
| Coloration | ≤GY1* | — | GY3.1 | GY3.1 | GY4.1 | GY1.8 | GY1.8 | GY1.8 | GY1.9 | GY1.9 | GY1.9 |
| pH | 3.0-4.4 | — | 3.9 | 3.9 | 3.9 | 3.9 | 3.9 | 3.9 | 3.9 | 3.9 | 3.9 |
| Osmolality | 2520-3080 | mOsm/kg | 2764 | 2752 | 2776 | 2775 | 2748 | 2747 | 2770 | 2777 | 2776 |
| Particulates | ≤6000/vial | — | 20 | 7 | 20 | 33 | 7 | 7 | 0 | 40 | 113 |
| (≥10 μm) | | | | | | | | | | | |
| Particulates | ≤600/vial | — | 0 | 0 | 7 | 0 | 0 | 0 | 0 | 7 | 60 |
| (>25 μm) | | | | | | | | | | | |
| Aluminum | <250 | μg/L | nt | nt | nt | nt | nt | nt | <20** | <20** | <20** |
| Assay | | | | | | | | | | | |
| Iron | 1.8-2.2 | μmol/mL | 1.9 | 1.9 | 1.9 | 2.0 | 2.0 | 1.9 | 2.0 | 2.0 | 1.9 |
| Zinc | 6.9-8.5 | μmol/mL | 7.5 | 7.4 | 7.4 | 7.5 | 7.4 | 7.4 | 7.5 | 7.5 | 7.4 |
| Manganese | 0.09-0.11 | μmol/mL | 0.10 | 0.10 | 0.10 | 0.10 | 0.10 | 0.10 | 0.10 | 0.10 | 0.10 |
| Copper | 0.54-0.66 | μmol/mL | 0.59 | 0.59 | 0.59 | 0.60 | 0.59 | 0.59 | 0.59 | 0.59 | 0.59 |
| Selenium | 0.09-0.11 | μmol/mL | 0.10 | 0.10 | 0.10 | 0.10 | 0.10 | 0.10 | 0.10 | 0.10 | 0.10 |
| Molybdenum | 0.018-0.022 | μmol/mL | 0.020 | 0.020 | 0.020 | 0.020 | 0.020 | 0.020 | 0.020 | 0.020 | 0.020 |
| Chromium | 0.018-0.022 | μmol/mL | 0.019 | 0.019 | 0.019 | 0.020 | 0.019 | 0.019 | 0.019 | 0.020 | 0.019 |
| Iodide | 0.09-0.11 | μmol/mL | 0.10 | 0.10 | 0.10 | 0.10 | 0.10 | 0.10 | 0.10 | 0.10 | 0.10 |
| Fluoride | 4.5-5.5 | μmol/mL | 5.0 | 5.0 | 5.0 | 5.0 | 5.0 | 5.0 | 4.9 | 4.9 | 4.9 |

*Not more intensively colored than reference solution GY1;
nt, not tested;
**inverted sample

TABLE 20
9 Months Room (25° C. ± 2° C./40% RH ± 5% RH) or Intermediate
(30° C. ± 2° C./65% RH ± 5% RH) Temperature, Upright

| | Acceptance | | Room Temperature | | | Intermediate | | |
|---|---|---|---|---|---|---|---|---|
| Test | criteria | Units | Batch1 | Batch2 | Batch3 | Batch1 | Batch2 | Batch3 |
| Clarity | Clear | — | Clear | Clear | Clear | Clear | Clear | Clear |
| Coloration | <GY1* | — | GY1.9 | GY1.9 | GY1.9 | GY1.9 | GY1.9 | GY1.9 |
| pH | 3.0-4.4 | — | 3.9 | 3.9 | 3.9 | 3.9 | 3.9 | 3.9 |
| Osmolality | 2520-3080 | mOsm/kg | 2769 | 2769 | 2759 | 2779 | 2744 | 2752 |
| Particulates | ≤6000/vial | — | 0 | 0 | 33 | 13 | 13 | 13 |
| (≥10 μm) | | | | | | | | |
| Particulates | ≤600/vial | — | 0 | 0 | 0 | 0 | 0 | 0 |
| (>25 μm) | | | | | | | | |
| Aluminum | <250 | μg/L | nt | nt | nt | nt | nt | nt |
| Assay | | | | | | | | |
| Iron | 1.8-2.2 | μmol/mL | 2.0 | 1.9 | 1.9 | 2.0 | 1.9 | 2.0 |
| Zinc | 6.9-8.5 | μmol/mL | 7.5 | 7.5 | 7.5 | 7.5 | 7.5 | 7.5 |
| Manganese | 0.09-0.11 | μmol/mL | 0.10 | 0.10 | 0.10 | 0.10 | 0.10 | 0.10 |
| Copper | 0.54-0.66 | μmol/mL | 0.59 | 0.59 | 0.59 | 0.59 | 0.59 | 0.59 |
| Selenium | 0.09-0.11 | μmol/mL | 0.10 | 0.10 | 0.10 | 0.10 | 0.10 | 0.10 |
| Molybdenum | 0.018-0.022 | μmol/mL | 0.020 | 0.020 | 0.020 | 0.020 | 0.020 | 0.020 |
| Chromium | 0.018-0.022 | μmol/mL | 0.019 | 0.019 | 0.019 | 0.019 | 0.019 | 0.020 |
| Iodide | 0.09-0.11 | μmol/mL | 0.10 | 0.10 | 0.10 | 0.10 | 0.10 | 0.10 |
| Fluoride | 4.5-5.5 | μmol/mL | 5.1 | 5.0 | 5.0 | 4.9 | 4.8 | 4.8 |

*Not more intensively colored than reference solution GY1;
nt, not tested

These results show that a trace element formulation according to the invention packaged in a 100-mL vial is stable under room temperature, intermediate, and accelerated storage conditions for at least six to nine months.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A sterile, injectable formulation comprising at least one trace element selected from zinc, iron, manganese, copper, selenium, molybdenum, iodine, chromium, and fluorine, or a pharmaceutically acceptable salt of the foregoing; a chelating agent, optionally an acid and/or a base as needed to adjust the pH, and water, wherein the formulation has a pH of about 3.0-4.5.

2. The formulation of claim 1, comprising a pharmaceutically acceptable salt of each of the following trace elements: zinc, iron, manganese, copper, selenium, molybdenum, iodine, chromium, and fluorine.

3. The formulation of claim 1, comprising:
from about 200 mg/L to about 20 g/L zinc sulfate heptahydrate,
from about 50 mg/L to about 5000 mg/L ferric chloride hexahydrate,
from about 2 mg/L to about 200 mg/L manganese chloride tetrahydrate,
from about 10 mg/L to about 1000 mg/L copper chloride dihydrate,
from about 2 mg/L to about 200 mg/L sodium selenite,
from about 0.5 mg/L to about 50 mg/L sodium molybdate dihydrate,
from about 2 mg/L to about 200 mg/L potassium iodide,
from about 0.5 mg/L to about 50 mg/L chromic chloride hexahydrate,
from about 20 mg/L to about 2000 mg/L sodium fluoride,
wherein the chelating agent comprises a polycarboxylic acid, a conjugate base, and a sugar alcohol.

4. The formulation of claim 3, wherein the polycarboxylic acid and conjugate base comprise from about 0.2 g/L to about 20 g/L trisodium citrate dihydrate and from about 50 mg/L to about 5 g/L citric acid monohydrate.

5. The formulation of claim 3, wherein the sugar alcohol comprises from about 30 g/L to about 3000 g/L xylitol.

6. A sterile, injectable formulation comprising:
about 2215 mg/L zinc sulfate heptahydrate,
about 540 mg/L ferric chloride hexahydrate,
about 19.8 mg/L manganese chloride tetrahydrate,
about 102.3 mg/L copper chloride dihydrate,
about 17.3 mg/L sodium selenite,
about 4.9 mg/L sodium molybdate dihydrate,
about 16.6 mg/L potassium iodide,
about 5.3 mg/L chromic chloride hexahydrate, and
about 210 mg/L sodium fluoride.

7. The formulation of claim 6, comprising a chelating agent.

8. The formulation of claim 7, wherein the chelating agent comprises a polycarboxylic acid, a conjugate base, and a sugar alcohol.

9. The formulation of claim 7, wherein the formulation has a pH of about 3.0-4.5.

10. The formulation claim 8, wherein the polycarboxylic acid and conjugate base comprise about 2134 mg/L trisodium citrate dihydrate and about 475 mg/L citric acid monohydrate, and the sugar alcohol comprises about 300 g/L xylitol.

11. A pharmaceutical product comprising a container and a formulation contained therein, wherein the formulation comprises chromium, copper, fluoride, iodine, iron, manganese, molybdenum, selenium, and zinc; a chelating agent, optionally an acid and/or a base as needed to adjust the pH, and water, wherein the formulation has a pH of about 3.0-4.5.

12. The pharmaceutical product of claim 11, wherein the chelating agent comprises a polycarboxylic acid, a conjugate base, and a sugar alcohol.

13. The pharmaceutical product of claim 11, comprising about 1 mcg/mL chromium, about 38 mcg/mL copper, about 95 mcg/mL fluoride, about 13 mcg/mL iodine, about 0.11 mg/mL iron, about 5.5 mcg/mL manganese, about 1.9 mcg/mL molybdenum, about 7.9 mcg/mL selenium, and about 0.5 mg/mL zinc.

14. The pharmaceutical product of claim 11, wherein about 10 mL or about 100 mL of the formulation is contained within the container, and wherein the container is a polypropylene vial.

15. The pharmaceutical product of claim 11, wherein the zinc is provided as a sulfate salt.

16. The pharmaceutical product of claim 11, wherein following storage of the product at room temperature for at least about 6 months, the formulation is clear and not more intensively colored than reference solution Greenish Yellow 1 (GY1) after storage of the product at room temperature for at least about 6 months.

17. The pharmaceutical product of claim 11, wherein the formulation comprises not more than about 250 mcg/L aluminum after storage of the product at room temperature for at least about 6 months.

18. The pharmaceutical product of claim 11, wherein the formulation is essentially free from visible particles and comprises, per container, not more than 6000 subvisible particles greater than or equal to 10 μm and not more than 600 subvisible particles greater than or equal to 25 μm after storage of the product at room temperature for about 6 months.

19. The pharmaceutical product of claim 11, wherein the formulation comprises 90%-110% of a labeled amount of each of the trace elements present in the formulation after storage of the product at room temperature for at least about 6 months.

20. The pharmaceutical product of claim 11, wherein the formulation does not contain any overage of iodide beyond a labeled amount.
